SEQUENCE LISTING (13 sequences)

SEQ ID NO: 1 - DNA, 357 bp, Homo sapiens
gaggtgcagctgctggagagcggcggagggctggtccagccaggcggcagcctgaggctgtcctgcgccgccagcggcttcaccttcagccactacgtgatggcctgggtgcggcaggccccaggcaagggcctggaatgggtgtccagcatcagcagcagcggcggctggaccctgtacgccgacagcgtgaagggcaggttcaccatcagcagggacaacagcaagaacaccctgtacctgcagatgaacagcctgagggccgaggacaccgccgtgtactactgcaccaggggcctgaagatggccaccatcttcgactactggggccagggcaccctggtgaccgtgagcagc

SEQ ID NO: 2 - protein, 119 aa, Homo sapiens [sequence omitted]

SEQ ID NO: 3 - DNA, 333 bp, Homo sapiens
cagtccgccctgacccagcccgccagcgtgagcggcagcccaggccagagcatcaccatcagctgcaccggcaccagcagcgacgtgggcagctacaacgtggtgtcctggtatcagcagcaccccggcaaggcccccaagctgatcatctacgaggtgtcccagaggcccagcggcgtgagcaacaggttcagcggcagcaagagcggcaacaccgccagcctgaccatcagcggcctgcagaccgaggacgaggccgactactactgctgcagctacgccggcagcagcatcttcgtgatcttcggcggagggaccaaggtgaccgtccta

SEQ ID NO: 4 - protein, 111 aa, Homo sapiens [sequence omitted]

SEQ ID NO: 5 - protein, 5 aa, Homo sapiens
HYVMA

SEQ ID NO: 6 - protein, 17 aa, Homo sapiens
SISSSGGWTLYADSVKG

SEQ ID NO: 7 - protein, 10 aa, Homo sapiens
GLKMATIFDY

SEQ ID NO: 8 - protein, 14 aa, Homo sapiens
TGTSSDVGSYNVVS

SEQ ID NO: 9 - protein, 7 aa, Homo sapiens
EVSQRPS

SEQ ID NO: 10 - protein, 11 aa, Homo sapiens
CSYAGSSIFVI

SEQ ID NO: 11 - protein, 1321 aa, Homo sapiens
SEVGNSQAVCPGTLNGLSVTGDAENQYQTLYKLYERCEVVMGNLEIVLTGHNADLSFLQWIREVTGYVLVAMNEFSTLPLPNLRVVRGTQVYDGKFAIFVMLNYNTNSSHALRQLRLTQLTEILSGGVYIEKNDKLCHMDTIDWRDIVRDRDAEIVVKDNGRSCPPCHEVCKGRCWGPGSEDCQTLTKTICAPQCNGHCFGPNPNQCCHDECAGGCSGPQDTDCFACRHFNDSGACVPRCPQPLVYNKLTFQLEPNPHTKYQYGGVCVASCPHNFVVDQTSCVRACPPDKMEVDKNGLKMCEPCGGLCPKACEGTGSGSRFQTVDSSNIDGFVNCTKILGNLDFLITQGDPWHKIPALDPEKLNVFRTVREITGYLNIQSWPPHMHNFSVFSNLTTIGGRSLYNRGFSLLIMKNLNVTSLGFRSLKEISAGRIYISANRQLCYHHSLNWTKVLRGPTEERLDIKHNRPRRDCVAEGKVCDPLCSSGGCWGPGPGQCLSCRNYSRGGVCVTHCNFLNGEPREFAHEAECFSCHPECQPMEGTATCNGSGSDTCAQCAHFRDGPHCVSSCPHGVLGAKGPIYKYPDVQNECRPCHENCTQGCKGPELQDCLGQTLVLIGKTHLTMALTVIAGLVVIFMMLGGTFLYWRGRRIQNKRAMRRYLERGESIEPLDPSEKANKVLARIFKETELRSLKVLGSGVFGTVHKGVWIPEGESIKIPVCIKVIEDKSGRQSFQAVTDHMLAIGSLDHAHIVRLLGLCPGSSLQLVTQYLPLGSLLDHVRQHRGALGPQLLLNWGVQIAKGMYYLEEHGMVHRNLAARNVLLKSPSQVQVADFGVADLLPPDDKQLLYSEAKTPIKWMALESIHFGKYTHQSDVWSYGVTVWELMTFGAEPYAGLRLAEVPDLLEKGERLAQPQICTIDVYMVMVKCWMIDENIRPTFKELANEFTRMARDPPRYLVIKRESGPGIAPGPEPHGLTNKKLEEVELEPELDLDLDLEAEEDNLATTTLGSALSLPVGTLNRPRGSQSLLSPSSGYMPMNQGNLGESCQESAVSGSSERCPRPVSLHPMPRGCLASESSEGHVTGSEAELQEKVSMCRSRSRSRSPRPRGDSAYHSQRHSLLTPVTPLSPPGLEEEDVNGYVMPDTHLKGTPSSREGTLSSVGLSSVLGTEEEDEDEEYEYMNRRRRHSPPHPPRPSSLEELGYEYMDVGSDLSASLGSTQSCPLHPVPIMPTAGTTPDEDYEYMNRQRDGGGPGGDYAAMGACPASEQGYEEMRAFQGPGHQAPHVHYARLKTLRSLEATDSAFDNPDYWHSRLFPKANAQRT

SEQ ID NO: 12 - protein, 445 aa, Homo sapiens
EVQLLESGGGLVQPGGSLRLSCAASGFTFSHYVMAWVRQAPGKGLEWVSSISSSGGWTLYADSVKGRFTISRDNSKNTLYLQMNSLRAEDTAVYYCTRGLKMATIFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 13 - protein, 217 aa, Homo sapiens
QSALTQPASVSGSPGQSITISCTGTSSDVGSYNVVSWYQQHPGKAPKLIIYEVSQRPSGVSNRFSGSKSGNTASLTISGLQTEDEADYYCCSYAGSSIFVIFGGGTKVTVLGQPKAAPSVTLFPPSSEELQANKATLVCLVSDFYPGAVTVAWKADGSPVKVGVETTKPSKQSNNKYAASSYLSLTPEQWKSHRSYSCRVTHEGSTVEKTVAPAECS

(19) United States
(12) Patent Application Publication
(10) Pub. No.: US 2014/0248280 A1
(43) Pub. Date: Sep. 4, 2014

(54) ANTI-ERBB3 ANTIBODIES IN COMBINATION WITH PACLITAXEL FOR TREATMENT OF GYNECOLOGICAL CANCERS

(71) Applicants: William Kubasek, Belmont, MA (US); Victor Moyo, Ringoes, NJ (US); Joseph Pearlberg, Jamaica Plain, MA (US); Isabelle Tabah-Fisch, Boulogne (FR); Gavin MacBeath, Wakefield, MA (US)

(72) Inventors: William Kubasek, Belmont, MA (US); Victor Moyo, Ringoes, NJ (US); Joseph Pearlberg, Jamaica Plain, MA (US); Isabelle Tabah-Fisch, Boulogne (FR); Gavin MacBeath, Wakefield, MA (US)

(73) Assignees: SANOFI, Paris (FR); MERRIMACK PHARMACEUTICALS, INC., Cambridge, MA (US)

(21) Appl. No.: 14/130,058

(22) PCT Filed: Jun. 13, 2012
(86) PCT No.: PCT/US12/42164

§ 371 Date: Apr. 14, 2014

Related U.S. Application Data
(60) Provisional application No. 61/503,342, filed on Jun. 30, 2011.
(60) Provisional application No. 61/529,630, filed on Aug. 31, 2011.
(60) Provisional application No. 61/596,102, filed on Feb. 7, 2012.

(30) Foreign Application Priority Data
Jan. 30, 2012 (FR) 1250860

(51) Int. Cl. A61K 39/395; A61K 31/337

(52) U.S. Cl. CPC A61K 39/3955; A61K 31/337
USPC 424/158.1

(57) ABSTRACT

Provided are methods and compositions for clinical treatment of advanced gynecological cancers using anti-ErbB3 antibodies combined with paclitaxel.

### CROSS-REFERENCE TO RELATED APPLICATIONS

This application claims the benefit of priority of U.S. Provisional Application No. 61/503,342 filed Jun. 30, 2011, U.S. Provisional Application No. 61/529,630 filed Aug. 31, 2011, U.S. Provisional Application No. 61/596,102 filed Feb. 7, 2012, and French Application No. 1250860, filed Jan. 30, 2012, all of which are incorporated herein by reference.

### BACKGROUND

Despite improvements in cancer therapies and late-stage options, there remains a critical need to optimize established therapies and develop new, promising therapies which prolong patients' lives while maintaining a high quality of life, particularly in the case of advanced cancers which are resistant or refractory to existing therapies.

The ErbB3 receptor is 148 kD transmembrane receptor belonging to the ErbB/EGFR receptor tyrosine kinase family although lacks intrinsic kinase activity. The ErbB receptors form homo- and heterodimeric complexes that impact the physiology of cells and organs by mediating ligand-dependent (and in some cases ligand independent) activation of multiple signal transduction pathways. ErbB3-containing heterodimers (such as ErbB2/ErbB3) in tumor cells have been shown to be the most mitogenic and oncogenic receptor complex within the ErbB family. Upon binding of heregulin (HRG), a physiological ligand for the ErbB3 receptor, ErbB3 dimerizes with other ErbB family members, predominantly ErbB2. ErbB3/ErbB2 dimerization results in transphosphorylation of ErbB3 on tyrosine residues contained within the cytoplasmic tail of the protein. Phosphorylation of these sites creates SH2 docking sites for SH2-containing proteins, including PI3-kinase. ErbB3-containing heterodimeric complexes are therefore potent activators of AKT, as ErbB3 possesses six tyrosine phosphorylation sites with YXXM motifs that, when phosphorylated, serve as excellent binding sites for phosphoinositol-3-kinase (PI3K), the action of which results in subsequent downstream activation of the AKT pathway. These six PI3K sites serve as a strong amplifier of ErbB3 signaling. Activation of this pathway further elicits several important biological processes involved in tumorigenesis, such as cell growth, migration and survival.

Heregulin has been shown to be involved in several different types of cancer: breast, ovarian, endometrial colon, gastric, lung, thyroid, glioma, medulloblastoma, melanoma as well as head and neck squamous cell carcinoma. In most of these tumor types, HRG regulates growth, invasion and angiogenesis through either over expression or the activation of an autocrine or paracrine loop. Disruption of the heregulin autocrine loop by blocking HRG binding or disruption of the ErbB2/ErbB3 dimer may provide an important therapeutic approach to controlling cancer cell growth.

### SUMMARY

Provided are compositions and methods for treating gynecological cancer in a human patient, comprising administering to the patient a combination of an anti-ErbB3 antibody and paclitaxel, wherein the combination is administered (or is for administration) according to a particular clinical dosage regimen (i.e., at a particular dose amount and according to a specific dosing schedule). Preferably, the human patient has gynecological cancer. In one embodiment the gynecological cancer is an advanced cancer. In another embodiment the gynecological cancer is resistant or refractory to treatment with platinum-based agents. In various such embodiments, the gynecological cancer is locally advanced or metastatic epithelial ovarian cancer, recurrent ovarian cancer, fallopian tube cancer or primary peritoneal cancer.

An exemplary anti-ErbB3 antibody is Antibody A or antigen binding fragments and variants thereof. In one embodiment, the antibody comprises variable heavy (VH) and/or variable light (VL) regions encoded by the nucleic acid sequences set forth in SEQ ID NOs:1 and 3, respectively. In another embodiment, the antibody comprises VH and/or VL regions comprising the amino acid sequences set forth in SEQ ID NOs 2 and 4, respectively. In another embodiment, the Antibody A comprises (in amino-to carboxy-terminal order) CDRH1, CDRH2, and CDRH3 sequences comprising the amino acid sequences set forth in SEQ ID NO: 5 (CDRH1) SEQ ID NO: 6 (CDRH2) and SEQ ID NO: 7 (CDRH3), and/or (in amino-to carboxy-terminal order) CDRL1, CDRL2, and CDRL3 sequences comprising the amino acid sequences set forth in SEQ ID NO: 8 (CDRL1) SEQ ID NO: 9 (CDRL2) and SEQ ID NO: 10 (CDRL3). In another embodiment, an antibody is used that competes for binding with and/or binds to the same epitope on human ErbB3 as the above-mentioned antibodies. In a particular embodiment, the epitope comprises residues 92-104 of human ErbB3 (SEQ ID NO: 11). In another embodiment, the antibody competes with Antibody A for binding to human ErbB3 and has at least 90% variable region amino acid sequence identity with the above-mentioned anti-ErbB3 antibodies. See, e.g., U.S. Pat. No. 7,846,440 and US Patent Publication No. 20100266584.

Accordingly, in one aspect, methods for treatment (e.g., effective treatment) of an advanced gynecological cancer in a human patient are provided, the methods comprising: administering to the patient, an effective amount of (a) an anti-ErbB3 antibody comprising CDRH1, CDRH2, and CDRH3 sequences comprising the amino acid sequences set forth in SEQ ID NO: 5 (CDRH1) SEQ ID NO: 6 (CDRH2) and SEQ ID NO: 7 (CDRH3), and CDRL1, CDRL2, and CDRL3 sequences comprising the amino acid sequences set forth in SEQ ID NO: 8 (CDRL1) SEQ ID NO: 9 (CDRL2) and SEQ ID NO: 10 (CDRL3), and (b) paclitaxel, wherein the method comprises at least one cycle, wherein the cycle is a period of 4 weeks, wherein for each cycle the anti-ErbB3 antibody is administered at a weekly dose of 20 mg/kg except for cycle 1 week 1 wherein the anti-ErbB3 antibody optionally may be administered at 40 mg/kg, and the paclitaxel is administered at a dose of 80 mg/m² once per week, and wherein the gynecological cancer is selected from the group consisting of locally advanced or metastatic epithelial ovarian cancer, recurrent ovarian cancer, fallopian tube cancer and primary peritoneal cancer. In one embodiment, the anti-ErbB3 antibody is Antibody A.

In another embodiment, after two cycles an alternate dosing cycle is administered, wherein for each alternate dosing cycle the anti-ErbB3 antibody is administered at a weekly dose of 20 mg/kg and the paclitaxel is administered at a dose of 80 mg/m² once per week for the first three weeks of the alternate dosing cycle, and is not administered during the fourth week of the alternate dosing cycle.

In another aspect, methods for treatment of an advanced gynecological cancer in a human patient are provided, the methods comprising: administering to the patient, an effective amount of (a) an anti-ErbB3 antibody comprising CDRH1, CDRH2, and CDRH3 sequences comprising the amino acid sequences set forth in SEQ ID NO: 5 (CDRH1), SEQ ID NO: 6 (CDRH2), and SEQ ID NO: 7 (CDRH3), and CDRL1, CDRL2, and CDRL3 sequences comprising the amino acid sequences set forth in SEQ ID NO: 8 (CDRL1), SEQ ID NO: 9 (CDRL2), and SEQ ID NO: 10 (CDRL3), and (b) paclitaxel, wherein the method comprises at least one cycle, wherein the cycle is a period of 4 weeks, wherein for each cycle the anti-ErbB3 antibody is administered at a weekly dose of 12 mg/kg, except for cycle 1 week 1 wherein the anti-ErbB3 antibody optionally may be administered at 20 mg/kg, and the paclitaxel is administered at a dose of 80 mg/m² once per week, and wherein the gynecological cancer is selected from the group consisting of locally advanced or metastatic epithelial ovarian cancer, recurrent ovarian cancer, fallopian tube cancer and primary peritoneal cancer.

In yet another aspect, methods for treatment of an advanced gynecological cancer in a human patient are provided, the methods comprising: administering to the patient, an effective amount of (a) an anti-ErbB3 antibody comprising CDRH1, CDRH2, and CDRH3 sequences comprising the amino acid sequences set forth in SEQ ID NO: 5 (CDRH1), SEQ ID NO: 6 (CDRH2), and SEQ ID NO: 7 (CDRH3), and CDRL1, CDRL2, and CDRL3 sequences comprising the amino acid sequences set forth in SEQ ID NO: 8 (CDRL1), SEQ ID NO: 9 (CDRL2), and SEQ ID NO: 10 (CDRL3), and (b) paclitaxel, wherein the method comprises at least one cycle, wherein the cycle is a period of 4 weeks, wherein for each cycle the anti-ErbB3 antibody is administered every other week at a dose of 20 mg/kg and the paclitaxel is administered at a dose of 80 mg/m² once per week, and wherein the gynecological cancer is selected from the group consisting of locally advanced or metastatic epithelial ovarian cancer, recurrent ovarian cancer, fallopian tube cancer and primary peritoneal cancer.

In still another aspect, methods for treatment of an advanced gynecological cancer in a human patient are provided, the methods comprising: administering to the patient, an effective amount of (a) an anti-ErbB3 antibody comprising CDRH1, CDRH2, and CDRH3 sequences comprising the amino acid sequences set forth in SEQ ID NO: 5 (CDRH1), SEQ ID NO: 6 (CDRH2), and SEQ ID NO: 7 (CDRH3), and CDRL1, CDRL2, and CDRL3 sequences comprising the amino acid sequences set forth in SEQ ID NO: 8 (CDRL1), SEQ ID NO: 9 (CDRL2), and SEQ ID NO: 10 (CDRL3), and (b) paclitaxel, wherein the method comprises at least one cycle, wherein the cycle is a period of 4 weeks, wherein for each cycle the anti-ErbB3 antibody is administered every other week at a dose of 40 mg/kg and the paclitaxel is administered at a dose of 80 mg/m² once per week, and wherein the gynecological cancer is selected from the group consisting of locally advanced or metastatic epithelial ovarian cancer, recurrent ovarian cancer, fallopian tube cancer and primary peritoneal cancer.

In one embodiment, the patient has been treated previously with a platinum-based compound.

In another embodiment, the anti-ErbB3 antibody is administered as a monotherapy prior to said at least one cycle.

In another embodiment, the monotherapy is administered at 40 mg/kg for one week.

In another embodiment, the cancer is platinum-resistant or refractory. In one embodiment, the cancer is resistant/refractory to cisplatin.

In another embodiment, the antibody is formulated for intravenous administration at a dose of 20 mg/kg.

In yet another aspect, kits for treating gynecological cancer in a human patient are provided, the kits comprising: a dose of an anti-ErbB3 antibody comprising CDRH1, CDRH2, and CDRH3 sequences comprising the amino acid sequences set forth, respectively, in SEQ ID NO: 5 (CDRH1) SEQ ID NO: 6 (CDRH2) and SEQ ID NO: 7 (CDRH3), and CDRL1, CDRL2, and CDRL3 sequences comprising the amino acid sequences set forth, respectively, in SEQ ID NO: 8 (CDRL1) SEQ ID NO: 9 (CDRL2) and SEQ ID NO: 10 (CDRL3), and instructions for using the anti-ErbB3 antibody in the method of claim 1.

In one embodiment, a kit of the invention comprises at least 500 mg of the antibody.

In another embodiment, a kit of the invention comprise at least 1 mg of paclitaxel.

In another aspect, an antiErbB3 antibody is provided, the antibody comprising: SEQ ID NO: 5 (CDRH1), SEQ ID NO: 6 (CDRH2), SEQ ID NO: 7 (CDRH3), SEQ ID NO: 8 (CDRL1), SEQ ID NO: 9 (CDRL2), and SEQ ID NO: 10 (CDRL3), for co-administration with paclitaxel in at least one cycle, wherein the cycle is a period of four weeks, and wherein for each cycle the anti-ErbB3 antibody is administered at a weekly dose of 20 mg/kg, except for cycle 1 week 1 wherein the anti-ErbB3 antibody optionally may be administered at 40 mg/kg, and the paclitaxel is administered at a weekly dose of 80 mg/m².

### BRIEF DESCRIPTION OF THE DRAWINGS

FIG. 1 shows the response of cancer patients receiving combination therapy of Antibody A and paclitaxel. Cohort 1: Antibody A 20 mg/kg (loading), 12 mg/qw QW; paclitaxel 80 mg/m2. Cohort 2: Antibody A 40 mg/kg (loading), 20 mg/qw QW; paclitaxel 80 mg/m2. Exp. Cohort 1: Antibody A 40 mg/kg (loading), 20 mg/qw QW; paclitaxel 80 mg/m2. Exp. Cohort 2: Antibody A 20 mg/kg (loading), 12 mg/qw QW; paclitaxel 80 mg/m2. Exp. Cohort 3: Antibody A 40 mg/kg QOW; paclitaxel 80 mg/m2.

FIG. 2 shows the response of cancer patients receiving combination therapy of Antibody A and paclitaxel presented in terms of sum diameters (%).

FIG. 3 shows a schematic diagram of the phase 2 clinical trial prior to interim analysis.

FIG. 4 shows the study design following interim analysis.

### DETAILED DESCRIPTION

### I. Definitions

As used herein, the term “subject” or “patient” is a human cancer patient.

As used herein, “effective treatment” refers to treatment producing a beneficial effect, e.g., amelioration of at least one symptom of a disease or disorder. A beneficial effect can take the form of an improvement over baseline, i.e., an improvement over a measurement or observation made prior to initiation of therapy according to the method. A beneficial effect can also take the form of arresting, slowing, retarding, or stabilizing of a deleterious progression of a marker of gynecological cancer. Effective treatment may refer to alleviation of at least one symptom of gynecological cancer Such effective treatment may, e.g., reduce patient pain, reduce the size and/or number of lesions, may reduce or prevent metastasis of a tumor, and/or may slow tumor growth.

The term “effective amount” refers to an amount of an agent that provides the desired biological, therapeutic, and/or prophylactic result. That result can be reduction, amelioration, palliation, lessening, delaying, and/or alleviation of one or more of the signs, symptoms, or causes of a disease, or any other desired alteration of a biological system. In reference to cancers, an effective amount comprises an amount sufficient to cause a tumor to shrink and/or to decrease the growth rate of the tumor (such as to suppress tumor growth) or to prevent or delay other unwanted cell proliferation. In some embodiments, an effective amount is an amount sufficient to delay tumor development. In some embodiments, an effective amount is an amount sufficient to prevent or delay tumor recurrence. An effective amount can be administered in one or more administrations. The effective amount of the drug or composition may: (i) reduce the number of cancer cells; (ii) reduce tumor size; (iii) inhibit, retard, slow to some extent and may stop cancer cell infiltration into peripheral organs; (iv) inhibit (i.e., slow to some extent and may stop tumor metastasis; (v) inhibit tumor growth; (vi) prevent or delay occurrence and/or recurrence of tumor; and/or (vii) relieve to some extent one or more of the symptoms associated with the cancer. In one example, an “effective amount” is the amount of Antibody A and the amount of paclitaxel clinically proven to effect a significant decrease in gynecological cancer or slowing of progression of gynecological cancer, such as platinum resistant/refractory advanced ovarian cancer.

The term “antibody” describes polypeptides comprising at least one antibody derived antigen binding site (e.g., VH/VL region or Fv, or complementarity determining region—CDR) that specifically binds to ErbB3. Antibodies include known forms of antibodies. For example, the antibody can be a human antibody, a humanized antibody, a bispecific antibody, or a chimeric antibody. The antibody also can be a Fab, Fab′2, ScFv, SMIP, Affibody®, nanobody, or a domain antibody. The antibody also can be of any of the following isotypes: IgG1, IgG2, IgG3, IgG4, IgM, IgA1, IgA2, IgAsec, IgD, and IgE. The antibody may be a naturally occurring antibody or may be an antibody that has been altered (e.g., by mutation, deletion, substitution, conjugation to a non-antibody moiety). For example, an antibody may to include one or more variant amino acids (compared to a naturally occurring antibody) which changes a property (e.g., a functional property) of the antibody. For example, numerous such alterations are known in the art which affect, e.g., half-life, effector function, and/or immune responses to the antibody in a patient. The term antibody\also includes artificial polypeptide constructs which comprise at least one antibody-derived antigen binding site.

Paclitaxel is a natural product with antitumor activity. The drug is produced via a semi-synthetic process from Taxus baccata. The chemical name for Paclitaxel is (5β,20-Epoxy-1,2α,4,7β,10β,13α-hexahydroxytax-11-en-9-one 4,10-diacetate 2-benzoate 13-ester with (2R,3S)-N-benzoyl-3-phenylisoserine. Paclitaxel is sold under the trade name Taxol®.

As used herein the terms “platinum-based agent” or “platinum therapy” refer to organoplatinum compounds, including for example carboplatin and cisplatin.

As used herein the term “resistant” refers to tumor cells that may have responded to a chemotherapeutic agent initially, but that became resistant during treatment. The term “refractory” as used herein refers to tumor cells that did not respond to the chemotherapeutic agent or continued to grow in the presence of the chemotherapeutic agent. In one embodiment a resistant or refractory tumor is one where, the treatment-free interval following completion of a course of therapy for a patient having the tumor is less than 6 months (e.g., owing to recurrence of the cancer) or where there is tumor progression during the course of therapy.

### II. Anti-ErbB3 Antibodies

Useful anti-ErbB3 antibodies (or VH/VL domains derived therefrom) can be made using methods well known in the art. Alternatively, art recognized anti-ErbB3 antibodies can be used. For example, Ab#3, Ab #14, Ab #17, Ab #19, described in U.S. Pat. No. 7,846,440, can be used. Antibodies that compete with any of these antibodies for binding to ErbB3 also can be used. Additional art-recognized anti-ErbB3 antibodies which can be used include those disclosed in U.S. Pat. No. 7,285,649; US20200310557; US20100255010, as well as antibodies IB4C3 and 2D1D12 (U3 Pharma Ag), both of which are described in e.g., US2004/0197332; anti-ErbB3 antibody referred to as AMG888 (U3-1287-U3 Pharma Ag and Amgen); and monoclonal antibody 8B8, described in U.S. Pat. No. 5,968,511. One example of such an antibody is Antibody A having heavy and light chains comprising the amino acid sequences set forth in SEQ ID NOs 12 and 13, respectively. Antibody A is referred to as “Ab #6” in U.S. Pat. No. 7,846,440.

In one embodiment, the anti-ErbB3 antibody comprises variable heavy (VH) and/or variable light (VL) regions encoded by the nucleic acid sequences set forth in SEQ ID NOs:1 and 3, respectively. In another embodiment, the antibody comprises VH and/or VL regions comprising the amino acid sequences set forth in SEQ ID NOs: 2 and 4, respectively. In another embodiment, the antibody comprises CDRH1, CDRH2, and CDRH3 sequences comprising the amino acid sequences set forth in SEQ ID NO: 5 (CDRH1) SEQ ID NO: 6 (CDRH2) and SEQ ID NO: 7 (CDRH3), and/or CDRL1, CDRL2, and CDRL3 sequences comprising the amino acid sequences set forth in SEQ ID NO: 8 (CDRL1) SEQ ID NO: 9 (CDRL2) and SEQ ID NO: 10 (CDRL3). In another embodiment, the antibody competes for binding with and/or binds to the same epitope on human ErbB3 as the above-mentioned antibodies. In a particular embodiment, the epitope comprises residues 92-104 of human ErbB3 (SEQ ID NO: 11). In another embodiment, the antibody binds to human ErbB3 and has at least 90% variable region sequence identity with the above-mentioned antibodies.

In other embodiments, the antibody is a fully human monoclonal antibody, such as an IgG2, that binds to ErbB3 and prevents the HRG and EGF-like ligand-induced intracellular phosphorylation of ErbB3.

Anti-ErbB3 antibodies, such as Antibody A, can be generated, e.g., in prokaryotic or eukaryotic cells, using methods well know in the art. In one embodiment, the antibody is produced in a cell line capable of glycosylating proteins, such as CHO cells.

### III. Pharmaceutical Compositions

Pharmaceutical compositions suitable for administration to a patient are typically in forms suitable for parenteral administration, e.g., in a in liquid carrier, or suitable for reconstitution into liquid solution or suspension, for intravenous administration.

In general, compositions typically comprise a pharmaceutically acceptable carrier. As used herein, the term “pharmaceutically acceptable” means approved by a government regulatory agency or listed in the U.S. Pharmacopeia or another generally recognized pharmacopeia for use in animals, particularly in humans. The term “carrier” refers to a diluent, adjuvant, excipient, or vehicle with which the compound is administered. Such pharmaceutical carriers can be sterile liquids, such as water and oils, including those of petroleum, animal, vegetable or synthetic origin, such as peanut oil, soybean oil, mineral oil, sesame oil, glycerol polyethylene glycol ricinoleate, and the like. Water or aqueous solution saline and aqueous dextrose and glycerol solutions may be employed as carriers, particularly for injectable solutions (e.g., comprising an anti-ErbB3 antibody). Liquid compositions for parenteral administration can be formulated for administration by injection or continuous infusion. Routes of administration by injection or infusion include intravenous, intraperitoneal, intramuscular, intrathecal and subcutaneous. In one embodiment, both anti-ErbB3 antibody and paclitaxel are administered intravenously (e.g., separately or together, each, e.g., over the course of one hour).

Antibody A for intravenous infusion (e.g., over the course of one hour) is supplied as a clear liquid solution in sterile, single-use vials containing 10.1 ml of Antibody A at a concentration of 25 mg/ml in an aqueous solution of 20 mM histidine, 150 mM sodium chloride, pH 6.5, which should be stored at 2-8° C.

Paclitaxel injection, USP is a clear colorless to slightly yellow viscous solution. It is supplied as a nonaqueous solution intended for dilution with a suitable parenteral fluid prior to intravenous infusion. Paclitaxel is available in 30 mg (5 mL), 100 mg (16.7 mL), and 300 mg (50 mL) multidose vials. Each mL of sterile nonpyrogenic solution contains 6 mg Paclitaxel, 527 mg of polyoxyl 35 castor oil, NF1 and 49,7% (v/v) dehydrated alcohol, USP.

Paclitaxel has the following structural formula:

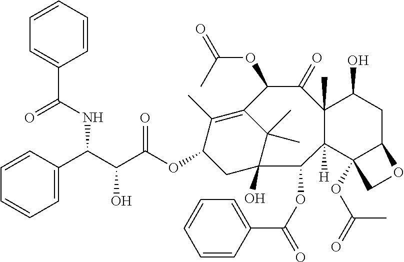 [SMILES: CC(=O)O[C@H]1C(=O)[C@@]2(C)[C@H]([C@H](OC(=O)c3ccccc3)[C@]3(O)C[C@H](OC(=O)[C@H](O)[C@@H](NC(=O)c4ccccc4)c4ccccc4)C(C)=C1C3(C)C)[C@]1(OC(C)=O)CO[C@@H]1C[C@@H]2O]

Paclitaxel is a white to off-white crystalline powder with the molecular formula C471151 NO14 and a molecular weight of 8519. It is highly lipophilic, insoluble in water, and melts at around 216° C. to 217° C.

### IV. Patient Populations

Provided herein are effective methods for treating certain gynecological cancers in a human patient using a combination of an anti-ErbB3 antibody and paclitaxel. In one embodiment, a human patient for treatment using the subject methods and compositions has locally advanced/metastatic or recurrent epithelial ovarian cancer. In another embodiment, a human patient for treatment using the subject methods and compositions has fallopian tube cancer. In one embodiment, a human patient for treatment using the subject methods and compositions has primary peritoneal cancer.

In one embodiment, a human patient for treatment using the subject methods and compositions has evidence of recurrent or persistent disease following primary chemotherapy.

In another embodiment, a human patient for treatment using the subject methods and compositions has had at least one prior platinum based chemotherapy regimen for management of primary or recurrent disease, e.g., a chemotherapy regimen comprising carboplatin, cisplatin, or another organoplatinum compound.

In another embodiment, the patient has a gynecological cancer that is platinum-resistant or refractory. In one example, the platinum-resistant/refractory cancer is ovarian cancer.

In another embodiment, the cancer undergoing treatment is advanced. In one aspect, the term “advanced” cancer denotes a cancer above Stage II. In another, “advanced” refers to a stage of disease where chemotherapy is typically recommended, which is any one of the following: 1. in the setting of recurrent disease: any stage or grade; 2. stage IC or higher, any grade; 3. stage IA or IB, grade 2 or 3; or 4. in the setting of incomplete surgery or suspected residual disease after surgery (where further surgery can not be performed): any stage or grade.

Patients can be tested or selected for one or more of the above described clinical attributes prior to, during or after treatment.

### V. Combination Therapy

As herein provided, anti-ErbB3 antibodies are administered adjunctively with paclitaxel, to effect improvement in subjects having certain gynecological cancer. In one embodiment, the anti-ErbB3 antibody is Antibody A.

As used herein, adjunctive or combined administration (coadministration) includes simultaneous administration of the compounds in the same or different dosage form, or separate administration of the compounds (e.g., sequential administration). For example, the antibody can be simultaneously administered with paclitaxel, wherein both the antibody and paclitaxel are formulated together. Alternatively, the antibody can be administered in combination with the paclitaxel, wherein both the antibody and paclitaxel are formulated for separate administration and are administered concurrently or sequentially. For example, the antibody can be administered first followed by the administration of the paclitaxel, or vice versa. Such concurrent or sequential administration preferably results in both Antibody A and paclitaxel being simultaneously present in treated patients.

In another embodiment, anti-ErbB3 antibody is formulated for intravenous administration. In particular embodiments, the anti-ErbB3 antibody is administered at a dose selected from: of 40 mg/kg, 20 mg/kg, 12 mg/kg, 10 mg/kg, 6 mg/kg, and/or 3.2 mg/kg. In one embodiment, the dose of antibody is varied over time. For example, the antibody may be initially administered at a high dose and may be lowered over time. In another embodiment, the antibody is initially administered at a low dose and increased over time. In another embodiment, a dose of 40 mg/kg of Antibody A is administered once per week for one or two weeks, followed by a dose of 20 mg/kg of Antibody A in combination with Paclitaxel.

### VI. Treatment Protocols

Suitable treatment protocols include, for example, those wherein (A) the anti-ErbB3 antibody is administered to a patient (i.e., human subject) once per week over a course of four weeks (at a dose of 20 mg/kg), and (B) the paclitaxel is administered to a patient i) once per week for four weeks or ii) once per week for the first three weeks of the anti-ErbB3 treatment and not during the fourth week. The cycle of treatment is four weeks.

In one embodiment, the cycle is repeated every four weeks.

In one embodiment, the anti-ErbB3 antibody is administered as a monotherapy prior to at least one cycle of anti-ErbB3 antibody/paclitaxel combination therapy. In one embodiment, anti-ErbB3 antibody monotherapy is administered for one week. In another embodiment, anti-ErbB3 antibody monotherapy is administered for two weeks, wherein the anti-ErbB3 antibody is administered at i) 40 mg/kg for two weeks or ii) 40 mg/kg the first week and at 20 mg/kg the second week.

In one embodiment, paclitaxel is administered in combination with an amount of Antibody A at an interval measured of at least seven days. A suitable weekly dosage of paclitaxel is 80 mg/m².

In another embodiment, a total of four doses of Antibody A are administered four times in a 4-week cycle, i.e., one dose per week. The administration cycle can be repeated, as necessary.

In another embodiment, the amount of Antibody A antibody administered is constant for each dose. In another embodiment, the amount of antibody administered varies with each dose. For example, the maintenance (or follow-on) dose of the antibody can be higher or the same as the loading dose which is first administered. In another embodiment, the maintenance dose of the antibody can be lower or the same as the loading dose.

### VII. Outcomes

With respect to target lesions, responses to therapy may include:

Complete Response (CR): Disappearance of all target lesions. Any pathological lymph nodes (whether target or non-target) must have reduction in short axis to <10 mm;
Partial Response (PR): At least a 30% decrease in the sum of the diameters of target lesions, taking as reference the baseline sum diameters;
Progressive Disease (PD): At least a 20% increase in the sum of the diameters of target lesions, taking as reference the smallest sum on study (this includes the baseline sum if that is the smallest on study). In addition to the relative increase of 20%, the sum must also demonstrate an absolute increase of at least 5 mm. (Note: the appearance of one or more new lesions is also considered progression); and
Stable Disease (SD): Neither sufficient shrinkage to qualify for PR nor sufficient increase to qualify for PD, taking as reference the smallest sum diameters while on study. (Note: a change of 20% or less that does not increase the sum of the diameters by 5 mm or more is coded as stable disease). To be assigned a status of stable disease, measurements must have met the stable disease criteria at least once after study entry at a minimum interval of 6 weeks.
With respect to non-target lesions, responses to therapy may include:
Complete Response (CR): Disappearance of all non-target lesions and normalization of tumor marker level. All lymph nodes must be non-pathological in size (<10 mm short axis). If tumor markers are initially above the upper normal limit, they must normalize for a patient to be considered in complete clinical response;
Non-CR/Non-PD: Persistence of one or more non-target lesion(s) and/or maintenance of tumor marker level above the normal limits; and
Progressive Disease (PD): Appearance of one or more new lesions and/or unequivocal progression of existing non-target lesions. Unequivocal progression should not normally trump target lesion status. It must be representative of overall disease status change, not a single lesion increase.

In exemplary outcomes, patients treated according to the methods disclosed herein may experience improvement in at least one sign of gynecological cancer, such as platinum resistant/refractory advanced ovarian cancer.

In one embodiment, the patient so treated exhibits CR, PR, or SD.

In another embodiment, the patient so treated experiences tumor shrinkage and/or decrease in growth rate, i.e., suppression of tumor growth. In another embodiment, unwanted cell proliferation is reduced or inhibited. In yet another embodiment, one or more of the following can occur: the number of cancer cells can be reduced; tumor size can be reduced; cancer cell infiltration into peripheral organs can be inhibited, retarded, slowed, or stopped; tumor metastasis can be slowed or inhibited; tumor growth can be inhibited; recurrence of tumor can be prevented or delayed; one or more of the symptoms associated with cancer can be relieved to some extent.

In other embodiments, such improvement is measured by a reduction in the quantity and/or size of measurable tumor lesions. Measurable lesions are defined as those that can be accurately measured in at least one dimension (longest diameter is to be recorded) as >10 mm by CT scan (CT scan slice thickness no greater than 5 mm), 10 mm caliper measurement by clinical exam or >20 mm by chest X-ray. The size of non-target lesions, e.g., pathological lymph nodes can also be measured for improvement. In one embodiment, lesions can be measured on chest x-rays or CT or MRI films.

In other embodiments, cytology or histology can be used to evaluate responsiveness to a therapy. The cytological confirmation of the neoplastic origin of any effusion that appears or worsens during treatment when the measurable tumor has met criteria for response or stable disease can be considered to differentiate between response or stable disease (an effusion may be a side effect of the treatment) and progressive disease.

In some embodiments, administration of effective amounts of the anti-ErbB3 antibody and paclitaxel according to any of the methods provided herein produce at least one therapeutic effect selected from the group consisting of reduction in size of a tumor, reduction in number of metastatic lesions appearing over time, complete remission, partial remission, stable disease, increase in overall response rate, or a pathologic complete response. In some embodiments, the provided methods of treatment produce a comparable clinical benefit rate (CBR=CR+PR+SD≧6 months) better than that achieved by paclitaxel alone. In other embodiments, the improvement of clinical benefit rate is about 20% 20%, 30%, 40%, 50%, 60%, 70%, 80% or more compared to paclitaxel alone.

### VIII. Kits and Unit Dosage Forms

Also provided are kits that include a pharmaceutical composition containing an anti-ErbB3 antibody, such as Antibody A, and a pharmaceutically-acceptable carrier, in a therapeutically effective amount adapted for use in the preceding methods. The kits can optionally also include instructions, e.g., comprising administration schedules, to allow a practitioner (e.g., a physician, nurse, or patient) to administer the composition contained therein to administer the composition to a patient having gynecological cancer. In one embodiment, the kit further comprises paclitaxel. In another embodiment the kit includes a syringe.

Optionally, the kits include multiple packages of the single-dose pharmaceutical composition(s) each containing an effective amount of the antibody (e.g., Antibody A) for a single administration in accordance with the methods provided above. Optionally, instruments or devices necessary for administering the pharmaceutical composition(s) may be included in the kits. For instance, a kit may provide one or more pre-filled syringes containing an amount of Antibody A that is about 100 times the dose in mg/kg indicated for administration in the above methods. Optionally, the kit may further comprise paclitaxel in a desired unit dosage form (e.g., a unit dosage form distributed by the manufacturer of paclitaxel) for administration.

The following examples are merely illustrative and should not be construed as limiting the scope of this disclosure in any way as many variations and equivalents will become apparent to those skilled in the art upon reading the present disclosure.

All patents, patent applications and publications cited herein are incorporated herein by reference in their entireties.

### EXAMPLES

### Example 1

### Phase 1 Trial in Certain Gynecological and Breast Cancers

A phase 1 trial of Antibody A in combination with paclitaxel was conducted in patients with certain gynecological and breast cancers to evaluate the safety and tolerability of escalating doses of Antibody A antibody and paclitaxel, as well as to determine the maximum tolerated dose of Antibody A in combination with paclitaxel and to characterize dose-limiting toxicities associated with the combination.

In the study each of paclitaxel and Antibody A antibody were administered once per week. A fixed dose of paclitaxel was administered (80 mg/m2 once per week) in combination with Antibody A at a dose of either i) 12 mg/kg the first week followed by 6 mg/kg weekly thereafter, ii) 20 mg/kg the first week followed by 12 mg/kg weekly thereafter, or iii) 40 mg/kg the first week followed by 20 mg/kg weekly thereafter. One treatment cycle in the study consisted of weekly treatments for four weeks. Cycles were repeated every four weeks.

To participate in the phase 1 study, patients with locally advanced/metastatic or recurrent epithelial ovarian cancer, fallopian tube cancer, primary peritoneal cancer, endometrial cancer, or cytological or histological confirmation of locally advanced/metastatic Her2 non-overexpressing breast cancer were identified. Those patients with ovarian, fallopian, primary peritoneal or endometrial cancer had evidence of recurrent or persistent disease following primary chemotherapy and had received at least one prior platinum based chemotherapy regimen (or high dose therapy, consolidation treatment, or extended therapy delivered after surgical or non-surgical assessment). Those patients with ovarian, fallopian or primary peritoneal cancer were confirmed as having platinum-resistant or refractory cancer as described herein. Those patients with Her2 non-overexpressing breast cancer had evidence of recurrent or persistent disease following at least one prior therapy in the locally advanced or metastatic setting and were documented as having non Her2 overexpressing cancer (as demonstrated using methods known in the art e.g., negative by IHC staining of 0 or 1+, a FISH result of less than 4.0 Her2 gene copies per nucleus, or a FISH ratio of less than 1.8).

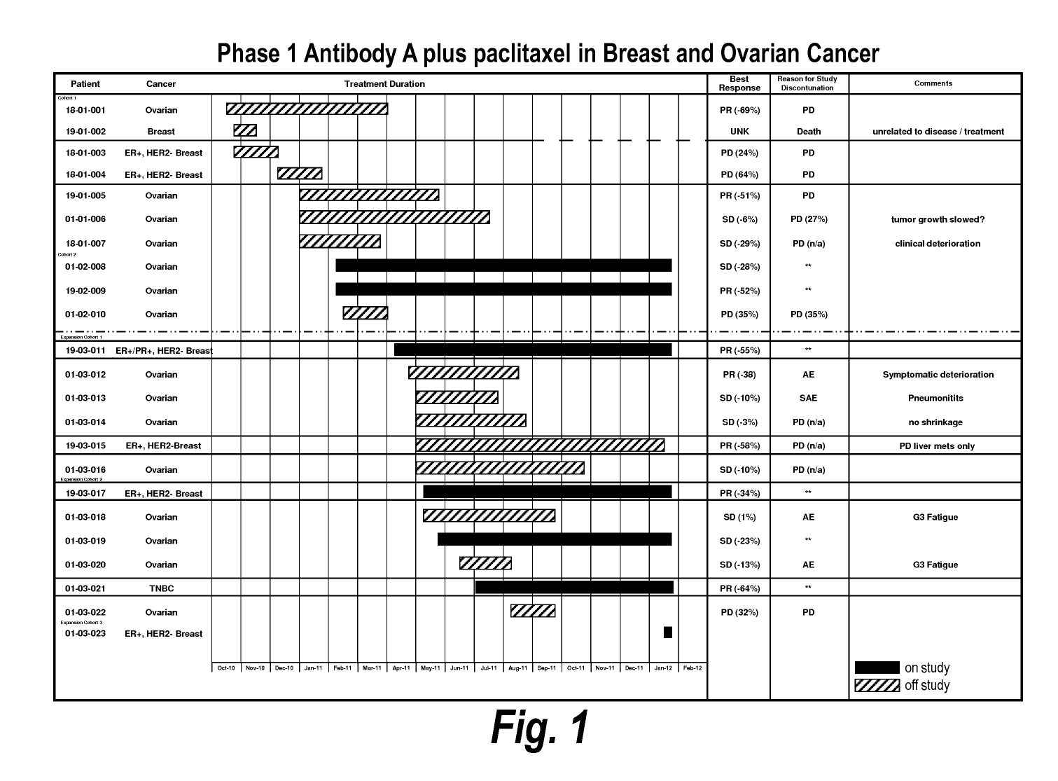

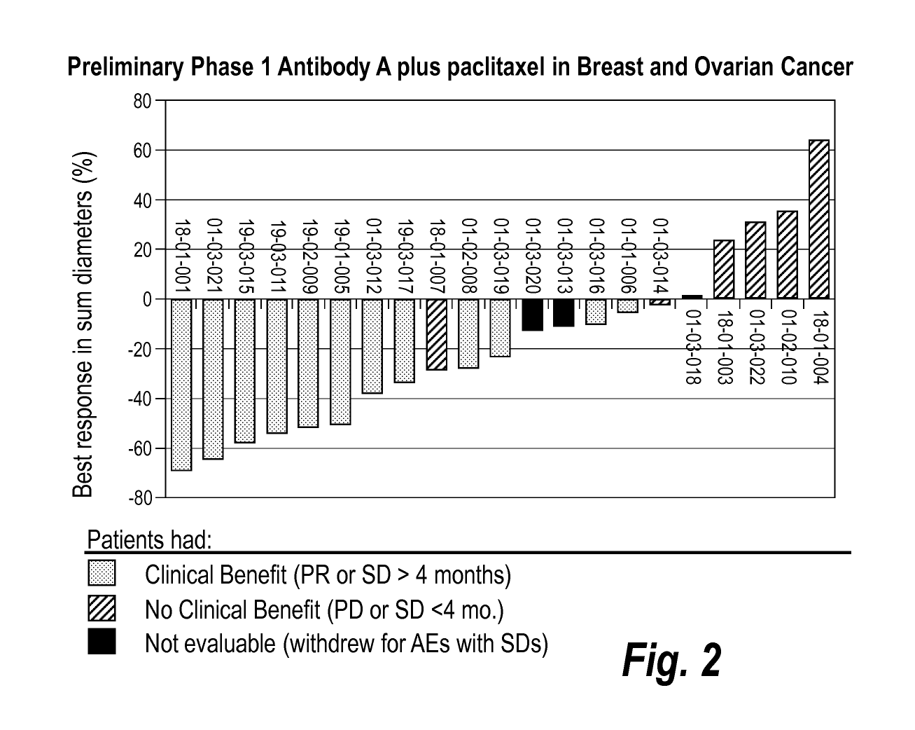

Summaries of preliminary responses are provided in FIGS. 1-2. As shown in FIG. 1, 13 patients with ovarian cancer who received combination therapy displayed a clinical benefit, as demonstrated by stable disease or partial response. As of May 2012, 24 patients have been treated with a median follow up of 5.5 months (range 0.8-13.1). The median age was 58 years (range 38-72), and patients had received a median of 4 (range 1-11) prior lines of therapy. Common (>20%) adverse events of any grade included fatigue (62%), peripheral neuropathy (58%), diarrhea (46%), neutropenia (46%) and rash (38%). Grade 3/4 toxicities included fatigue (17%), peripheral neuropathy (8%), diarrhea (12%), neutropenia (16%), anemia (4%), abdominal pain (8%), and hypokalemia (4%). 17 (71%) patients were evaluable for response and the overall clinical benefit rate, defined as PR or SD lasting for >4 months, was 71%. 47% achieved a PR and 35% a confirmed PR with a median duration of response of 4.6 months (range 1.7-9.6) and 24% had SD>4 months with a median duration of SD of 5.6 months (range 4.2-12.6). 17% patients had PD at first assessment and 38% remain on study with a median on-study time of 10.2 months (range 1.4-13.1).

Alternate dosing schedules will be examined in an expansion cohort of 24 patients, as described below in Table 2. Dose level 1 will be enrolled first. Once all six patients have been enrolled, dose level 2 can begin enrollment, followed by dose levels 3 and 4.

TABLE 2
Dosing Schedule of Antibody A/Paclitaxel for Expansion Cohort

| | Antibody A Dose | Paclitaxel Dose | # Patients |
|---|---|---|---|
| 1 | 40 mg/kg loading dose x1 (Cycle 1 | 80 mg/kg paclitaxel | 6 patients |
| | Week 1) followed by 20 mg/kg | QW | |
| | weekly maintenance dose | | |
| | thereafter | | |
| 2 | 20 mg/kg loading dose x1 (Cycle 1 | 80 mg/kg paclitaxel | 6 patients |
| | Week 1) followed by 12 mg/kg | QW | |
| | weekly maintenance dose | | |
| | thereafter | | |
| 3 | 40 mg/kg QOW of Antibody A | 80 mg/kg paclitaxel | 6 patients |
| | | QW | |
| 4 | 20 mg/kg QOW of Antibody A | 80 mg/kg paclitaxel | 6 patients |
| | | QW | |

Patients receiving QOW (every other week) dosing of Antibody A will not receive Antibody A on weeks 2 and 4 of each cycle.

### Example 2

### Phase 2 Trial in Certain Gynecological Cancers

A phase 2 trial of Antibody A in combination with paclitaxel is conducted in patients with certain gynecological cancers to demonstrate the efficacy of administering Antibody A as part of a combination treatment comprising paclitaxel.

### Objectives

The primary objectives of this study are to determine whether the combination of Antibody A plus paclitaxel is more effective than paclitaxel alone based on Progression Free Survival (PFS) in patients with advanced or metastatic ovarian cancers resistant or refractory to platinum agents and to correlate a pre-specified biomarker panel reflective of ErbB3 signalling activity with patient outcome (correlation between PFS and other clinical efficacy criteria with biomarker signature).

The secondary objectives of the study include:

i) comparing the efficacy of the combination of Antibody A plus paclitaxel to paclitaxel alone using: Overall survival, Objective response rate and duration of response, and Clinical benefit rate, defined as CR, PR and stabilization of disease lasting at least 6 months;

ii) to further characterize the safety profile of the Antibody A plus paclitaxel combination;

iii) to gather exploratory data on additional potentially predictive biomarkers to be measured in serum and tumor tissue; and iv) to determine the immunogenicity parameters of the Antibody A plus paclitaxel combination.

### Study Design

This is a multicenter, open-label, randomized, Phase II study of Antibody A in patients with advanced gynecological cancer, such as ovarian cancer resistant or refractory to platinum agents. Up to 210 patients are randomized (2:1) to receive Antibody A plus paclitaxel or paclitaxel alone. The randomization is stratified by patient's ECOG Performance Status (0-1 versus 2) and number of prior therapies (1 and 2 vs. 3+).

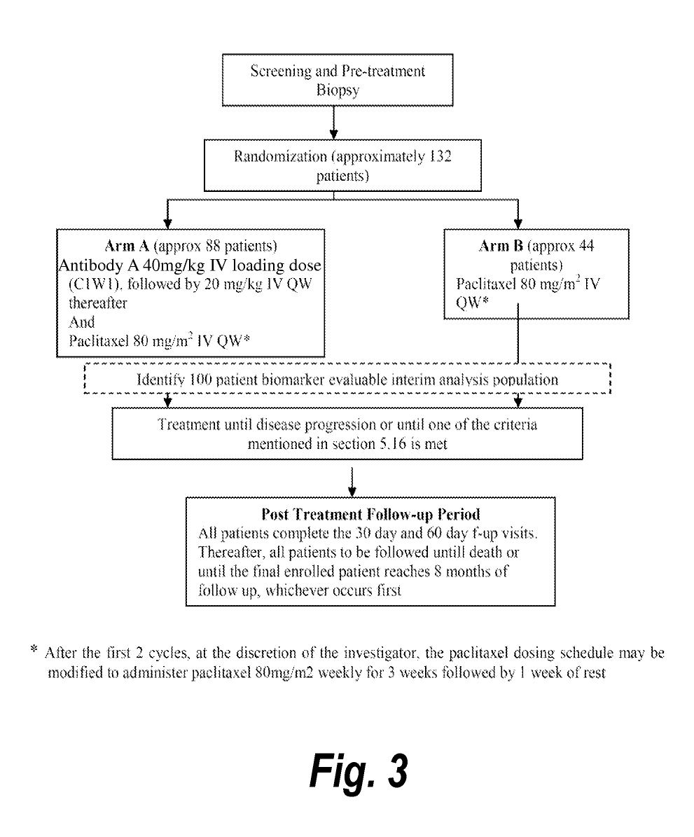

As shown in FIG. 3, arm A of the study, the experimental arm, administers: Antibody A 40 mg/kg IV loading dose for one week, followed by 20 mg/kg IV each week thereafter, and paclitaxel 80 mg/m2 IV each week. Arm B of the study, the control arm, administers Paclitaxel 80 mg/m2 IV each week.

Patients on the experimental arm (arm A) receive paclitaxel infusion immediately after the first dose of Antibody A on Cycle 1 Day 1. After the first 2 cycles, at the discretion of the investigator, the dosing schedule may be modified to administer 80 mg/m2 weekly for 3 weeks followed by 1 week of rest. The infusion should be prepared as directed in the paclitaxel package insert. All patients receiving paclitaxel may be pre-medicated as known in the art or as described herein.

One treatment cycle will consist of weekly treatments for 4 weeks. Cycles are repeated every 4 weeks until disease progression, intolerable toxicity or other reason for study termination. Patients are assessed for evidence of disease progression every 8 weeks from the date of first dose (Cycle 1 Day 1), or sooner.

Patients may undergo a core biopsy during screening, prior to the first dose of study drug. An optional second biopsy may also be completed at the time of disease progression. If available, archived tumor samples from a primary tumor biopsy will also be requested of all patients. The material is processed to obtain quantitative measurements of five biomarkers, chosen based on their mechanistic relationship to the activation state of ErbB3. Three of these biomarkers EGFR (ErbB1), Her2/Neu (ErbB2) and Her3 (ErbB3) are measured by quantitative immunohistochemistry. The other two biomarkers—betacellulin and heregulin (neuregulin-1) are assessed using reverse transcription polymerase chain reaction (RT-PCR) preferably quantitative real-time reverse transcription polymerase chain reaction (qRT-PCR). The values of these candidate biomarkers are quantified in the pre-treatment biopsy samples. Along with these five core biomarkers, additional signaling proteins or their transcripts are also quantified.

Patients with evaluable biomarkers (as determined by analysis of patient samples) may be used to define the interim analysis population. It is estimated that approximately 132 patients are accrued in order to obtain 100 patients with evaluable biomarkers.

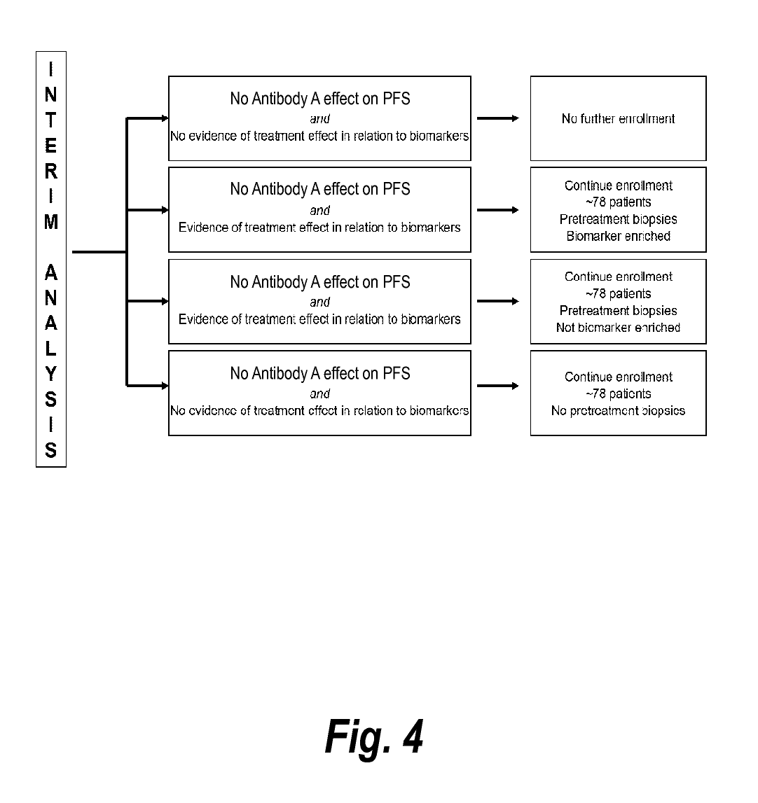

FIG. 3 illustrates the study design, and FIG. 4 illustrates the study design following interim analysis.

### Inclusion Criteria

For inclusion in the trial, patients will have/be: cytological or histological confirmation of locally advanced or metastatic epithelial ovarian cancer, recurrent epithelial ovarian cancer, fallopian tube cancer or primary peritoneal cancer; evidence of recurrent or persistent disease following primary chemotherapy; received at least one prior platinum based chemotherapy regimen for management of primary or recurrent disease (The platinum agent could have been carboplatin, cisplatin, or another organoplatinum compound. High dose therapy, consolidation treatment, or extended therapy delivered after surgical or non-surgical assessment also are permitted.); “Platinum-resistant or refractory” according to standard GOG criteria, defined as treatment-free interval following completion of platinum<6 months OR progression during platinum therapy; not received prior weekly paclitaxel therapy; and a negative pregnancy test prior to the study entry and be practicing an effective form of contraception if hysterectomy and/or oophorectomy were not part of the prior treatment (It is expected that the overwhelming majority of ovarian cancer patients would have had hysterectomy and oophorectomy as part of the original surgery.).

### Exclusion Criteria

Patients will meet all the inclusion criteria listed above and none of the following exclusion criteria: prior radiotherapy to >25% of the bone marrow-bearing areas; evidence of any other active malignancy; symptomatic CNS disease; active infection or an unexplained fever>38.5° C. during screening visits or on the first scheduled day of dosing; known hypersensitivity to any of the components of Antibody A or who have had hypersensitivity reactions to fully human monoclonal antibodies; received treatment, within 30 days prior to the first scheduled day of dosing, with any investigational agents that have not received regulatory approval for any indication or disease state; received other recent antitumor therapy including: investigational therapy administered within the 30 days prior to the first scheduled day of dosing in this study or radiation therapy or other standard systemic therapy within 14 days prior to the first scheduled dose in this study, including, in addition (if necessary), the timeframe for resolution of any actual or anticipated toxicities from such radiation; NYHA Class III or IV congestive heart failure or LVEF less than normal, per institutional guidelines, or <55%, if not otherwise specified by institutional guidelines (Patients with a significant history of cardiac disease, i.e. uncontrolled blood pressure, unstable angina, myocardial infarction within 1 year or ventricular arrhythmias requiring medication, also are excluded.); or history of severe allergic reactions to paclitaxel or other drugs formulated in Cremophor®EL, unless the patient has been desensitized in accordance with the institutional guidelines

### Dose Levels

Antibody A is administered weekly. Study drug should be brought to room temperature prior to administration. Vials of study drug should not be shaken. The appropriate quantity of study drug is removed from the vial, diluted in 250 mL of 0.9% normal saline and administered over 90 minutes, for the first infusion, and over 60 minutes for all subsequent infusions, using compatible infusion sets with a low protein binding 0.22 micrometer in-line filter.

A patient's body weight at the start of a cycle is to be used to calculate the dose used throughout the cycle. The first dose of Antibody A administered is 40 mg/kg. Following this loading dose, the subsequent weekly dose of Antibody A is 20 mg/kg. Should a patient's body weight change by 10%, a new total dose should be calculated to reflect this change.

Paclitaxel is a clear, colorless to slightly yellow viscous solution. It is supplied as a non aqueous solution intended for dilution with a suitable parenteral fluid prior to IV infusion. Paclitaxel is available in 30 mg (5 mL), 100 mg (16.7 mL), and 300 mg (50 mL) multidose vials. Each mL of sterile non pyrogenic solution contains 6 mg paclitaxel, 527 mg of purified Cremophor® EL (polyoxyethylated castor oil) and 49.7% (v/v) dehydrated alcohol, USP.

Vials of paclitaxel should be stored in the original cartons between 20° to 25° C. (68° to 77° F.) and protected from light.

Paclitaxel is administered at the dose of 80 mg/m² weekly, as an IV infusion over 60 minutes. For patients also receiving Antibody A, paclitaxel should be administered immediately following the Antibody A dose. All patients should be pre-medicated prior to paclitaxel administration in order to prevent severe hypersensitivity reactions. Such premedication may consist of 20 mg (orally) dexamethasone administered approximately 12 and 6 hours before paclitaxel, 50 mg (IV) diphenhydramine (or its equivalent) administered 30 to 60 minutes prior to paclitaxel, and 300 mg cimetidine (IV) or 50 mg ranitidine (IV) administered 30 to 60 minutes before paclitaxel.

### Dose Modification

Dose modification for paclitaxel can be made according to the table below:

TABLE 3
Dose Levels for Reduction of Paclitaxel Dose

| | | Dose | Dose |
|---|---|---|---|
| | | Reduction | Reduction |
| Drug | Starting Dose | Level A | Level B |
| Paclitaxel | 80 mg/m2 | 70 mg/m2 | 60 mg/m2 |

### Management of Toxicities

In the event of Grade 2 toxicities that are possibly related to Antibody A treatment, Investigators should use their discretion in deciding whether to continue the cycle of Antibody A dosing. However, patients should have recovered from toxicity to baseline or Grade 1 (except alopecia) prior to initiating treatment in the next cycle. If the time required for recovery from toxicity is more than 2 weeks, a patient's continuation on study should be discussed between investigator and sponsor regarding risks and benefits of continuation.

Paclitaxel therapy may be held up to 3 weeks to allow for recovery from toxicity related to paclitaxel. If a patient does not recover from toxicity within 3 weeks, they must discontinue paclitaxel treatment, but patients on Arm A, at the discretion of the Investigator, may continue Antibody A.

If a dose is reduced, it should remain reduced for the duration of the study, dose re-escalation to an earlier dose is not permitted.

### Pharmacokinetic Assessments

Serum levels of Antibody A and paclitaxel are measured via trough levels and at the end of infusion during various time points (section 10.5) in the first 30 patients at a central analytical lab using an ELISA based assay. If required, in order to better understand the PK and safety profile of Antibody A and Antibody A and paclitaxel combination, additional analytes may also be measured. Directions for processing and shipping the PK serum samples can be found in the study manual.

### Statistical Considerations

Patients are randomized in a 2:1 ratio to receive either Antibody A plus paclitaxel (Arm A) or paclitaxel alone (Arm B) according to a pre-specified randomization scheme.

The randomization is stratified by the following factors:

### ECOG Performance Status (0 and 1 vs 2)

Presence of metastasis (yes vs no)

### Study Design and Sample Size Determination

The study is implemented in a 2-stage manner. At the end first stage, an interim analysis is performed based on 100 (approximately 67 on Antibody A and paclitaxel arm and 33 on paclitaxel arm) biomarker evaluable patients to assess PFS and examine the predictive use of candidate biomarkers.

In order to assess whether any effect on PFS of Antibody A in combination with paclitaxel relative to paclitaxel alone is modified by selected biomarkers, the interaction between study treatment and each biomarker, as well as composite scores based on two or more biomarkers, are examined using a Cox proportional hazards model with PFS as outcome variable and treatment, biomarker and the cross-interaction between biomarker and treatment as covariates.

Antibody A treatment is considered active for the overall population at the interim if the log-rank test yields a p-value less than 0.20 (observed HR<0.75) and inactive otherwise. Interaction between biomarker/composite score and treatment is considered existing if the interaction test yields a p-value less than 0.20.

Four possible strategies for patient enrollment and tissue collection is considered:

1. No further enrollment. This may occur if there is insufficient evidence of an overall Antibody A PFS treatment effect and there is also insufficient evidence that the PFS treatment effect varies in relation to any selected biomarker.

In this scenario, the effective sample size for the biomarker evaluable population is 100 and the analysis has 62% power to detect a hazard ratio is 0.67 based on a 2-sided log-rank test at a significance level of 0.20.

The minimum number of patients required to obtain 100 biomarker evaluable patients is 100. However, biomarker data may only be obtained from a fraction of the enrolled patients. It is estimated that approximately 132 patients (in 2:1 allocation) are accrued in order to obtain 100 biomarker evaluable patients at the interim. The analysis of PFS in the overall population (n=132) at the interim with approximately 89 events will provide 71% power to detect a hazard ratio of 0.67 based on a 2-sided log-rank test at a significance level of 0.20.

2. Continue enrollment of approximately 78 additional patients within a biomarker-defined enriched subpopulation. This may occur if there is insufficient evidence of an overall Antibody A PFS treatment effect, but there is sufficient evidence that the PFS treatment effect varies in relation to one or more selected biomarker. In this case a post-hoc threshold is identified from the interim analysis for a selected biomarker or a composite score of multiple biomarkers based on predictive value to define enrichment.

Assuming that the biomarker prevalence is approximately 30%, roughly 30 biomarker evaluable patients are expected from the interim data (approximately 20 in the Antibody A treatment arm and 10 in paclitaxel alone arm). With an additional 78 patients allocated in 2:1 ratio, the total sample size for the enriched subpopulation is about 108 (approximately 72 in the Antibody A treatment arm and 36 in paclitaxel alone arm). In this biomarker enriched subpopulation, it is assumed that the median PFS is 9 months for patients treated with Antibody A plus paclitaxel and 6 months for patients treated with paclitaxel alone. With 84 PFS events in the subgroup, the final PFs analysis will have approximately 88% power to detect a hazard ratio of 0.57 based on a 2-sided log-rank test at a significance level of 0.20.

The actual enrollment for the overall population at the interim analysis may be different from the 132 patients as currently estimated. The actual prevalence of the biomarker-defined enriched subpopulation estimated from the interim data may also be much different from the assumed 30%. The number of additional enrollment may be modified accordingly. In any case, the total sample size in this scenario does exceed 210.

3. Continue enrollment of approximately 78 additional with pre-treatment biopsies, but independent of biomarker results. This may occur if there is sufficient evidence of an overall Antibody A PFS treatment effect and there is also sufficient evidence that the PFS treatment effect varies in relation to one or more selected biomarker or composite score. In this case a post-hoc threshold is identified from the interim analysis for a selected biomarker or a composite score of multiple biomarkers based on predictive value. The patients enrolled after the interim analysis are used to independently validate the predictive value of the identified biomarker.

The actual enrollment for the overall population at the interim analysis may be different from the 132 patients as currently estimated. The number of additional enrollment may be modified accordingly. The final sample size for the overall population is 210. With 164 PFS events the study will have approximately 88% power to detect a hazard ratio of 0.67 for the overall PFS treatment effect based on a 2-sided log-rank test at a significance level of 0.20.

Approximately 48 patients are expected in the biomarker enriched subpopulation under the assumption that about 75% of enrolled patients are biomarker evaluable and the prevalence of biomarker enriched population is about 30%. It is assumed that the median PFS is 10.5 months for the biomarker enriched subgroup patients treated with Antibody A plus paclitaxel and 6 months for patients treated with paclitaxel alone. With 34 PFS events out of 48 patients in the biomarker enriched subpopulation the study will have approximately 62% power to detect a hazard ratio of 0.57 based on a 2-sided log-rank test at a significance level of 0.20.

4. Continue enrollment of approximately 78 additional patients without pre-treatment biopsies. This may occur if there is sufficient evidence of an overall Antibody A PFS treatment effect, but there is insufficient evidence that the PFS treatment effect varies in relation to any selected biomarker.

The actual enrollment for the overall population at the interim analysis may be different from the 132 patients as currently estimated. The number of additional enrollment may be modified accordingly. The final sample size for the overall population is 210. With 164 PFS events, the final study will provide approximately 88% power to detect a hazard ratio of 0.67 based on a 2-sided log-rank test at a significance level of 0.20.

In summary, up to 210 patients are enrolled in the study and randomized in a 2:1 ratio to receive either Antibody A plus paclitaxel (Arm A) or paclitaxel alone (Arm B).

### Efficacy Analyses

The tumor assessment related to the efficacy endpoints (PFS, ORR, clinical benefit rate and overall survival) is analyzed using RECISTv1.1 and is evaluated based on Investigator assessment.

All efficacy analyses are performed for both the ITT and EP populations. No multiplicity adjustment is made.

### Primary Analysis of the Primary Endpoint

PFS is defined as the number of months from the date of randomization to the date of death or progression, whichever occurred earlier. If neither death nor progression is observed during the study, PFS data is censored at the last valid tumor assessment.

PFS is compared between the two treatment groups by the log-rank test procedure stratified by stratification factors as specified at the randomization.

The hazard ratio and corresponding 95% confidence interval is estimated using a Cox proportional hazard model stratified by the same stratification factors as those used for the log-rank test described above. The PFS curves are estimated using Kaplan-Meier estimates. In addition, different censoring and missing data imputing methods may be used to perform sensitivity analyses on PFS.

### Secondary Efficacy Endpoints and Analyses

### Overall Survival

Overall survival is defined as the time from patient randomization to death. Overall survival is compared between treatment arms using the stratified log-rank test and Kaplan-Meier curves of OS are presented. The hazard ratio and the associated 95% confidence interval are estimated using a Cox proportional hazards model stratified by ECOG performance status and number of prior therapies. In addition, the difference between the treatment groups with respect to the survival rate at 12 months and the 95% CI for the difference is presented.

### Objective Response Rate

The number and percentage of patients experiencing objective response (confirmed complete response (CR) or partial response (PR)) at the time of analysis is presented and the 95% confidence interval for the proportion is calculated. A stratified Mantel-Haenszel test is used for treatment comparison.

### Clinical Benefit Rate

The clinical benefit rate is defined as the proportion of patients who have stable disease (SD), PR or CR for at least 24 weeks. A stratified Mantel-Haenszel test is used for treatment comparison.

### Duration of Objective Response

The duration of objective response is defined as the date of first documented, confirmed objective response (CR or PR, whichever status is recorded first) to the earliest date that recurrent or progressive disease is objectively documented. If progression has not been documented, a patients duration of objective response is censored at the date of last assessment. Only patients who have achieved a confirmed response (CR or PR) are included in this analysis. The duration of objective response is analyzed using the Kaplan-Meier method. The median and 95% confidence interval is estimated and summarized by treatment group. A graph of the duration of response over time is presented.

### Safety Analysis

Treatment emergent adverse events are presented by treatment arm, by patient, by NCI CTCAE grade and by MedDRA system organ class (SOC). Adverse events, serious adverse events, adverse events related to Antibody A and Grade 3 and 4 adverse events are presented separately. Laboratory data are presented by treatment arm and by visit. Abnormal laboratory values are assessed according to NCI CTCAE grade, where possible. Evaluation of QTc is based upon Fridericias correction method. CTCAE criteria is applied to the QTcF (i.e. Grade 3=QTc>500 msec).

### Pharmacokinetics Analysis

Pharmacokinetic parameters are derived from the blood PK samples and are analyzed using descriptive statistics, including the median, mean and 95% confidence intervals around parameter estimates by dose level. PK parameters will include Cmax, Tmax, AUC (area under the concentration curve), clearance, volume of distribution at steady state (Vdss), and the terminal elimination half-life. Estimation of the pharmacokinetic parameters is performed using standard non-compartmental methods. Additional exploratory analysis may be performed on the PK sample, to help clarify any safety or PK issues related to Antibody A and/or paclitaxel that arise during the course of the study.

### Biomarker Analysis

### Tumor Samples

Tumor blocks or unstained slides containing tumor tissue from time of initial diagnosis are collected from each patient where available. In order to adequately evaluate the pharmacodynamics and the biomarkers predictive of response to the Antibody A combinations, direct sampling of the patients tumor will also be completed through core biopsies obtained prior to first dose administration, and at the time of progression (if the patient consents to a second biopsy). This material is processed to obtain quantitative measurements of five biomarkers, chosen based on their mechanistic relationship to the activation state of ErbB3. Three of these biomarkers—EGFR (ErbB1), Her2/Neu (ErbB2) and Her3 (ErbB3) are measured by quantitative immunohistochemistry. The other two biomarkers—Betacellulin and Heregulin (neuregulin-1) are assessed using reverse transcription polymerase chain reaction (RT-PCR). The values of these candidate biomarkers are quantified in the pre-treatment biopsy samples. Along with these five core biomarkers, additional signaling proteins or their transcripts are also quantified, using a variety of assay technologies including, but not limited to, immunohistochemistry, reverse-phase proteins arrays (RPPAs), quantitative mass spectrometry, RT-PCR, and DNA microarrays. These proteins or transcripts include other receptors, ligands, and downstream signaling proteins whose levels may vary with response to Antibody A. In addition, the levels of total ErbB3, pErbB3, pAKT, pERK, pS6, and other relevant pharmacodynamics markers, are analyzed in pre- and post-treatment samples. These tumor samples are analyzed as paired samples, when both pre- and on-treatment samples are available. The data are characterized using means and 95% confidence intervals for pre-treatment concentrations for the population and the mean and 95% confidence intervals for normalized change from baseline in paired samples.

### Blood Samples

Blood samples are collected to conduct exploratory studies to further characterize and correlate possible biomarkers that may help to predict or evaluate response to Antibody A. Samples are used to conduct specific biomarker analysis related to ErbB pathways or Antibody A mode of action. In order to assess whether any effect on PFS of Antibody A in combination with paclitaxel relative to paclitaxel alone is modified by selected biomarkers, the interaction between study treatment and each biomarker, as well as composite scores based on two or more biomarkers calculated using published methods (Schoeberl, et al, 2009Sci Signal 2:77 ra31 and Schoeberl et al, 2010 Cancer Res. 70:2485; 2494), are examined using a Cox proportional hazards model with PFS as outcome variable and treatment, biomarker and the cross-interaction between biomarker and treatment as covariates, using the data at the interim analysis.

If the interaction effect is significant at a significance level of 0.10 or is meaningful, then post-hoc threshold is determined and biomarker subgroup is identified. The power to detect a treatment by biomarker interaction at the interim analysis is assessed by simulations. It assumed that 66 PFS events will occur out of 100 biomarker evaluable patients. It is also assumed that biomarker values/composite scores (or transforms thereof) are normally distributed with common variance and that biomarkers are predictive of better progression-free survival only in the Antibody A treated arm. If a mean biomarker value differs between patients who are progression-free versus patients who are not at 6 months in the Antibody A treated arm by 1.5 standard deviations, then (assuming exponential PFS) the power to detect an interaction at a significance level of 0.10 is about 64%. If a mean value differs by 2 standard deviations, the power will increase to 69%. However, if the mean difference is as small as 1 standard deviation, then the power will drop to 50%.

### Interim Analysis

There is one interim analysis to occur when approximately 66 PFS events have been observed out of 100 biomarker evaluable patients. PFS are assessed and the biomarker-treatment interaction effect on PFS is examined. The Steering Committee will review the interim analysis results and determine the patient enrichment strategy for the second stage of the trial.

### Response Evaluation Criteria

For the purposes of this study, patients should be re-evaluated for response every 8 weeks (+/−1 week) from the date of first dose.

Response and progression is evaluated in this study using the international criteria proposed by the revised Response Evaluation Criteria in Solid Tumors (RECIST) guideline (version 1.1) [Eur J Ca 45:228-247, 2009]. Changes in the largest diameter (unidimensional measurement) of the tumor lesions and the shortest diameter in the case of malignant lymph nodes are used in RECIST.

The following general principles must be followed:
1. To assess objective response, it is necessary to estimate the overall tumor burden at baseline to which subsequent measurements are compared. All baseline evaluations should be performed as closely as possible to the beginning of treatment and not more than four weeks before registration.
2. Measurable disease is defined by the presence of at least one measurable lesion.
3. All measurements should be recorded in metric notation by use of a ruler or calipers.
4. The same method of assessment and the same technique must be used to characterize each identified lesion at baseline and during follow-up.

### Evaluable for Objective Response

Only those patients who have measurable disease present at baseline, have received at least one cycle of therapy, and have had their disease re-evaluated is considered evaluable for response. These patients will have their response classified according to the definitions stated below. (Note: Patients who exhibit objective disease progression prior to the end of cycle 1 will also be considered evaluable.)

### Evaluable Non-Target Disease Response

Patients who have lesions present at baseline that are evaluable but do not meet the definitions of measurable disease, have received at least one cycle of therapy, and have had their disease re-evaluated are considered evaluable for non-target lesion assessment. The response assessment is based on the presence, absence, or unequivocal progression of the lesions.

### Disease Parameters

### Measurable Disease

Measurable lesions are defined as those that can be accurately measured in at least one dimension (longest diameter to be recorded) as >20 mm by chest x-ray, as >10 mm with CT scan, or >10 mm with calipers by clinical exam. All tumor measurements are recorded in millimeters. Tumor lesions that are situated in a previously irradiated area are considered measurable if there is incontrovertible evidence of interval progression since completion of prior radiation, documented on relevant imaging.

### Malignant Lymph Nodes

To be considered pathologically enlarged and measurable, a lymph node is >15 mm in short axis when assessed by CT scan (CT scan slice thickness recommended to be no greater than 5 mm). At baseline and in follow-up, only the short axis is measured and followed.

### Non-Measurable Disease

All other lesions (or sites of disease), including small lesions (longest diameter<10 mm or pathological lymph nodes with >10 to <15 mm short axis), are considered non-measurable disease. Bone lesions, leptomeningeal disease, ascites, pleural/pericardial effusions, lymphangitis cutis/pulmonitis, inflammatory breast disease, and abdominal masses (not followed by CT or MRI), are considered as non-measurable. Non-measurable also includes lesions that are <20 mm by chest x-ray. Cystic lesions that meet the criteria for radiographically defined simple cysts should not be considered as malignant lesions (neither measurable nor non-measurable) since they are, by definition, simple cysts.

Cystic lesions thought to represent cystic metastases can be considered as measurable lesions, if they meet the definition of measurability described above. However, if non-cystic lesions are present in the same patient, these are preferred for selection as target lesions.

### Target Lesions

All measurable lesions up to a maximum of two lesions per organ and five lesions in total, representative of all involved organs, should be identified as target lesions and recorded and measured at baseline. Target lesions should be selected on the basis of their size (lesions with the longest diameter), be representative of all involved organs, but in addition should be those that lend themselves to reproducible repeated measurements. It may be the case that, on occasion, the largest lesion does not lend itself to reproducible measurement in which circumstance the next largest lesion which can be measured reproducibly should be selected.

A sum of the diameters (longest for non-nodal lesions, short axis for nodal lesions) for all target lesions is calculated and reported as the baseline sum diameters. If lymph nodes are to be included in the sum, then only the short axis is added into the sum. The baseline sum of the diameters is used as reference to further characterize any objective tumor regression in the measurable dimension of the disease.

### Non-Target Lesions

All other lesions (or sites of disease) including any measurable lesions over and above the five target lesions should be identified as non-target lesions and should also be recorded at baseline. Measurements of these lesions are not required, but the presence or absence of unequivocal progression of each should be noted throughout follow-up.

### Methods for Evaluation of Measurable Disease

All measurements should be taken and recorded in metric notation using a ruler or calipers. All baseline evaluations should be performed as closely as possible to the beginning of treatment and never more than 4 weeks before registration.

The same method of assessment and the same technique is used to characterize each identified and reported lesion at baseline and during follow-up. Imaging-based evaluation is preferred to evaluation by clinical examination unless the lesion(s) being followed cannot be imaged but are assessable by clinical exam.

### Clinical Lesions

Clinical lesions will only be considered measurable when they are superficial (e.g., skin nodules and palpable lymph nodes) and ≧10 mm in diameter as assessed using calipers (e.g., skin nodules). In the case of skin lesions, documentation by color photography, including a ruler to estimate the size of the lesion, is recommended.

### Chest X-Ray

Lesions on chest x-ray are acceptable as measurable lesions when they are clearly defined and surrounded by aerated lung. However, CT is preferable.

### Conventional CT and MRI

This guideline has defined measurability of lesions on CT scan based on the assumption that CT slice thickness is 5 mm or less. If CT scans have slice thickness greater than 5 mm, the minimum size for a measurable lesion should be twice the slice thickness. MRI is also acceptable in certain situations (e.g. for body scans). Use of MRI is complex. MRI has excellent contrast, spatial, and temporal resolution; however, there are many image acquisition variables involved in MRI which greatly impact image quality, lesion conspicuity, and measurement. Furthermore, the availability of MRI is variable globally. As with CT, if an MRI is performed, the technical specifications of the scanning sequences used should be optimized for the evaluation of the type and site of disease. Furthermore, as with CT, the modality used at follow-up must be the same as was used at baseline and the lesions should be measured/assessed on the same pulse sequence. It is beyond the scope of the RECIST guidelines to prescribe specific MRI pulse sequence parameters for all scanners, body parts, and diseases. Ideally, the same type of scanner should be used and the image acquisition protocol should be followed as closely as possible to prior scans. Body scans should be performed with breath-hold scanning techniques, if possible.

### PET-CT

At present, the low dose or attenuation correction CT portion of a combined PET-CT is not always of optimal diagnostic CT quality for use with RECIST measurements. However, if the site can document that the CT performed as part of a PET-CT is of identical diagnostic quality to a diagnostic CT (with IV and oral contrast), then the CT portion of the PET-CT can be used for RECIST measurements and can be used interchangeably with conventional CT in accurately measuring cancer lesions over time. Note, however, that the PET portion of the CT introduces additional data which may bias an investigator if it is not routinely or serially performed.

### Ultrasound

Ultrasound is not useful in assessment of lesion size and should not be used as a method of measurement. Ultrasound examinations cannot be reproduced in their entirety for independent review at a later date and, because they are operator dependent, it cannot be guaranteed that the same technique and measurements are taken from one assessment to the next. If new lesions are identified by ultrasound in the course of the study, confirmation by CT or MRI is advised. If there is concern about radiation exposure at CT, MRI may be used instead of CT in selected instances.

### Endoscopy, Laparoscopy

The utilization of these techniques for objective tumor evaluation is not advised. However, such techniques may be useful to confirm complete pathological response when biopsies are obtained or to determine relapse in trials where recurrence following complete response (CR) or surgical resection is an endpoint.

### Cytology, Histology

These techniques can be used to differentiate between partial responses (PR) and complete responses (CR) in rare cases (e.g., residual lesions in tumor types, such as germ cell tumors, where known residual benign tumors can remain). The cytological confirmation of the neoplastic origin of any effusion that appears or worsens during treatment when the measurable tumor has met criteria for response or stable disease is mandatory to differentiate between response or stable disease (an effusion may be a side effect of the treatment) and progressive disease.

### FDG-PET

While FDG-PET response assessments need additional study, it is sometimes reasonable to incorporate the use of FDG-PET scanning to complement CT scanning in assessment of progression (particularly possible ‘new’ disease). New lesions on the basis of FDG-PET imaging can be identified according to the following algorithm:

a. Negative FDG-PET at baseline, with a positive FDG-PET at follow-up is a sign of PD based on a new lesion, as long as there is clinical corroboratory evidence of a malignancy.

b. No FDG-PET at baseline and a positive FDG-PET at follow-up: If the positive FDG-PET at follow-up corresponds to a new site of disease confirmed by CT, this is PD. If the positive FDG-PET at follow-up is not confirmed as a new site of disease on CT, additional follow-up CT scans are needed to determine if there is truly progression occurring at that site (if so, the date of PD is the date of the initial abnormal FDG-PET scan). If the positive FDG-PET at follow-up corresponds to a pre-existing site of disease on CT that is not progressing on the basis of the anatomic images, this is not PD.

c. FDG-PET, if negative (−), may be used to upgrade a response to a CR in a manner similar to a biopsy in cases where a residual radiographic abnormality is thought to represent fibrosis or scarring. However, both approaches may lead to false positive CR due to limitations of FDGPET and biopsy resolution/sensitivity.

A “positive” FDG-PET scan lesion means one which is FDG avid with an uptake greater than twice that of the surrounding tissue on the attenuation corrected image.

### Response Criteria

### Evaluation of Target Lesions

### Complete Response (CR)

Disappearance of all target lesions. Any pathological lymph nodes (whether target or non-target) must have reduction in short axis to <10 mm.

### Partial Response (PR)

At least a 30% decrease in the sum of the diameters of target lesions, taking as reference the baseline sum diameters

### Progressive Disease (PD)

At least a 20% increase in the sum of the diameters of target lesions, taking as reference the smallest sum on study (this includes the baseline sum if that is the smallest on study). In addition to the relative increase of 20%, the sum must also demonstrate an absolute increase of at least 5 mm. (Note: the appearance of one or more new lesions is also considered progression).

### Stable Disease (SD)

Neither sufficient shrinkage to qualify for PR nor sufficient increase to qualify for PD, taking as reference the smallest sum diameters while on study. (Note: a change of 20% or less that does not increase the sum of the diameters by 5 mm or more is coded as stable disease) To be assigned a status of stable disease, measurements must have met the stable disease criteria at least once after study entry at a minimum interval of 6 weeks.

### Evaluation of Non-Target Lesions

### Complete Response (CR)

Disappearance of all non-target lesions and normalization of tumor marker level. All lymph nodes must be non-pathological in size (<10 mm short axis). If tumor markers are initially above the upper normal limit, they must normalize for a patient to be considered in complete clinical response.

### Non-CR/Non-PD

Persistence of one or more non-target lesion(s) and/or maintenance of tumor marker level above the normal limits.

### Progressive Disease (PD)

Appearance of one or more new lesions and/or unequivocal progression of existing non-target lesions. Unequivocal progression should not normally trump target lesion status. It must be representative of overall disease status change, not a single lesion increase.

When the patient also has measurable disease, there must be an overall level of substantial worsening in non-target disease such that, even in the presence of SD or PR in target disease, the overall tumor burden has increased sufficiently to merit discontinuation of therapy. A modest “increase” in the size of one or more non-target lesions is usually not sufficient to qualify for unequivocal progression status. The designation of overall progression solely on the basis of change in non-target disease in the face of SD or PR of target disease will therefore be extremely rare.

When the patient only has non-measurable disease, the increase in overall disease burden should be comparable in magnitude to the increase that would be required to declare PD for measurable disease: i.e., an increase in tumor burden from “trace” to “large”, an increase in nodal disease from “localized” to “widespread”, or an increase sufficient to require a change in therapy.

Although a clear progression of “non-target” lesions only is exceptional, the opinion of the treating physician should prevail in such circumstances, and the progression status should be confirmed at a later time by the review panel (or Principal Investigator).

### Evaluation of New Lesions

The appearance of new lesions constitutes Progressive Disease (PD).

### Evaluation of Best Overall Response

The best overall response is the best response recorded from the start of the treatment until disease progression/recurrence or non-protocol therapy (taking as reference for progressive disease the smallest measurements recorded since the treatment started). The patient's best response assignment will depend on the achievement of measurement criteria.

For Patients with Measurable Disease (i.e., Target Disease)

| Target | Non-Target | New | Best Overall | |
|---|---|---|---|---|
| Lesions | Lesions | Lesions* | Response | Remarks |
| CR | CR | No | CR | |
| CR | Non- | No | PR | |
| | CR/Non-PD | | | |
| CR | Not | No | PR | |
| | evaluated | | | |
| PR | Non-PD/not | No | PR | |
| | evaluated | | | |
| SD | Non-PD/not | No | SD | Documented |
| | evaluated | | | at least once >6 |
| | | | | weeks from |
| | | | | baseline |
| PD | Any | Yes or No | PD | No prior SD, |
| | | | | PR or CR |
| Any | PD** | Yes or No | PD | |
| Any | Any | Yes | PD | |

*See RECIST 1.1 manuscript for further details on what is evidence of a new lesion.
**In exceptional circumstances, unequivocal progression in non-target lesions may be accepted as disease progression.
Note:
Patients with a global deterioration of health status requiring discontinuation of treatment without objective evidence of disease progression at that time should be reported as “symptomatic deterioration.” Every effort should be made to document the objective progression even after discontinuation of treatment.

### Duration of Response

### Duration of Overall Response

The duration of overall response is measured from the time measurement criteria are met for CR or PR (whichever is first recorded) until the first date that recurrent or progressive disease is objectively documented (taking as reference for progressive disease the smallest measurements recorded since the treatment started).

The duration of overall CR is measured from the time measurement criteria are first met for CR until the first date that progressive disease is objectively documented.

### Duration of Stable Disease

Stable disease is measured from the start of the treatment until the criteria for progression are met, taking as reference the smallest measurements recorded since the treatment started, including the baseline measurements.

To be assigned a status of stable disease, measurements must have met the stable disease criteria at least once after study entry at a minimum interval of 6 weeks.

Those skilled in the art will recognize, and is able to ascertain and implement using no more than routine experimentation, many equivalents of the specific embodiments described herein. Such equivalents are intended to be encompassed by the following claims. Any combinations of the embodiments disclosed in the dependent claims are within the scope of the disclosure.

All patents, patent applications and publications cited herein are incorporated herein by reference in their entireties.

| | | SOURCE | | |
|---|---|---|---|---|
| SEQ | | OR | | |
| ID NO: | DESIGNATION | FORMAT | TYPE | SEQUENCE |
| 1 | Heavy Chain | Human | DNA | gaggtgcagc tgctggagag cggcggaggg |
| | Variable Region | VH | | ctggtccagc caggcggcag cctgaggctg |
| | (VH) of Antibody | | | tcctgcgccg ccagcggctt caccttcagc |
| | A | | | cactacgtga tggcctgggt gcggcaggcc |
| | | | | ccaggcaagg gcctggaatg ggtgtccagc |
| | | | | atcagcagca gcggcggctg gaccctgtac |
| | | | | gccgacagcg tgaagggcag gttcaccatc |
| | | | | agcagggaca acagcaagaa caccctgtac |
| | | | | ctgcagatga acagcctgag ggccgaggac |
| | | | | accgccgtgt actactgcac caggggcctg |
| | | | | aagatggcca ccatcttcga ctactggggc |
| | | | | cagggcaccc tggtgaccgt gagcagc |
| 2 | Heavy Chain | Human | PROTEIN | Glu Val Gln Leu Leu Glu Ser Gly Gly Gly |
| | Variable Region | VH | | Leu Val Gln Pro Gly Gly Ser Leu Arg Leu |
| | (VH) of Antibody | | | Ser Cys Ala Ala Ser Gly Phe Thr Phe Ser |
| | A | | | His Tyr Val Met Ala Trp Val Arg Gln Ala |
| | | | | Pro Gly Lys Gly Leu Glu Trp Val Ser Ser |
| | | | | Ile Ser Ser Ser Gly Gly Trp Thr Leu Tyr |
| | | | | Ala Asp Ser Val Lys Gly Arg Phe Thr Ile |
| | | | | Ser Arg Asp Asn Ser Lys Asn Thr Leu Tyr |
| | | | | Leu Gln Met Asn Ser Leu Arg Ala Glu Asp |
| | | | | Thr Ala Val Tyr Tyr Cys Thr Arg Gly Leu |
| | | | | Lys Met Ala Thr Ile Phe Asp Tyr Trp Gly |
| | | | | Gln Gly Thr Leu Val Thr Val Ser Ser |
| 3 | Light Chain | Human | DNA | cagtccgccc tgacccagcc cgccagcgtg |
| | Variable Region | VL | | agcggcagcc caggccagag catcaccatc |
| | (VL) of Antibody | | | agctgcaccg gcaccagcag cgacgtgggc |
| | A | | | agctacaacg tggtgtcctg gtatcagcag |
| | | | | caccccggca aggcccccaa gctgatcatc |
| | | | | tacgaggtgt cccagaggcc cagcggcgtg |
| | | | | agcaacaggt tcagcggcag caagagcggc |
| | | | | aacaccgcca gcctgaccat cagcggcctg |
| | | | | cagaccgagg acgaggccga ctactactgc |
| | | | | tgcagctacg ccggcagcag catcttcgtg |
| | | | | atcttcggcg gagggaccaa ggtgaccgtc cta |
| 4 | Light Chain | Human | PROTEIN | Gln Ser Ala Leu Thr Gln Pro Ala Ser Val |
| | Variable Region | VL | | Ser Gly Ser Pro Gly Gln Ser Ile Thr Ile |
| | (VL) of Antibody | | | Ser Cys Thr Gly Thr Ser Ser Asp Val Gly |
| | A | | | Ser Tyr Asn Val Val Ser Trp Tyr Gln Gln |
| | | | | His Pro Gly Lys Ala Pro Lys Leu Ile Ile |
| | | | | Tyr Glu Val Ser Gln Arg Pro Ser Gly Val |
| | | | | Ser Asn Arg Phe Ser Gly Ser Lys Ser Gly |
| | | | | Asn Thr Ala Ser Leu Thr Ile Ser Gly Leu |
| | | | | Gln Thr Glu Asp Glu Ala Asp Tyr Tyr Cys |
| | | | | Cys Ser Tyr Ala Gly Ser Ser Ile Phe Val |
| | | | | Ile Phe Gly Gly Gly Thr Lys Val Thr Val |
| | | | | Leu |
| 5 | Heavy Chain | Human | PROTEIN | His Tyr Val Met Ala |
| | CDR1 (CDRH1) | CDRH1 | | |
| | of Antibody A | | | |
| 6 | Heavy Chain | Human | PROTEIN | Ser Ile Ser Ser Ser Gly Gly Trp Thr Leu |
| | CDR2 (CDRH2) | CDRH2 | | Tyr Ala Asp Ser Val Lys Gly |
| | of Antibody A | | | |
| 7 | Heavy Chain | Human | PROTEIN | Gly Leu Lys Met Ala Thr Ile Phe Asp Tyr |
| | CDR3 (CDRH3) | CDRH3 | | |
| | of Antibody A | | | |
| 8 | Light Chain | Human | PROTEIN | Thr Gly Thr Ser Ser Asp Val Gly Ser Tyr |
| | CDR1 (CDRL1) | CDRL1 | | Asn Val Val Ser |
| | of Antibody A | | | |
| 9 | Light Chain | Human | PROTEIN | Glu Val Ser Gln Arg Pro Ser |
| | CDR2 (CDRL2) | CDRL2 | | |
| | of Antibody A | | | |
| 10 | Light Chain | Human | PROTEIN | Cys Ser Tyr Ala Gly Ser Ser Ile Phe Val |
| | CDR3 (CDRL3) | CDRL3 | | Ile |
| | of Antibody A | | | |
| 11 | Human ErbB3 | Human | PROTEIN | Ser Glu Val Gly Asn Ser Gln Ala Val Cys |
| | | | | Pro Gly Thr Leu Asn Gly Leu Ser Val Thr |
| | | | | Gly Asp Ala Glu Asn Gln Tyr Gln Thr Leu |
| | | | | Tyr Lys Leu Tyr Glu Arg Cys Glu Val Val |
| | | | | Met Gly Asn Leu Glu Ile Val Leu Thr Gly |
| | | | | His Asn Ala Asp Leu Ser Phe Leu Gln Trp |
| | | | | Ile Arg Glu Val Thr Gly Tyr Val Leu Val |
| | | | | Ala Met Asn Glu Phe Ser Thr Leu Pro Leu |
| | | | | Pro Asn Leu Arg Val Val Arg Gly Thr Gln |
| | | | | Val Tyr Asp Gly Lys Phe Ala Ile Phe Val |
| | | | | Met Leu Asn Tyr Asn Thr Asn Ser Ser His |
| | | | | Ala Leu Arg Gln Leu Arg Leu Thr Gln Leu |
| | | | | Thr Glu Ile Leu Ser Gly Gly Val Tyr Ile |
| | | | | Glu Lys Asn Asp Lys Leu Cys His Met Asp |
| | | | | Thr Ile Asp Trp Arg Asp Ile Val Arg Asp |
| | | | | Arg Asp Ala Glu Ile Val Val Lys Asp Asn |
| | | | | Gly Arg Ser Cys Pro Pro Cys His Glu Val |
| | | | | Cys Lys Gly Arg Cys Trp Gly Pro Gly Ser |
| | | | | Glu Asp Cys Gln Thr Leu Thr Lys Thr Ile |
| | | | | Cys Ala Pro Gln Cys Asn Gly His Cys Phe |
| | | | | Gly Pro Asn Pro Asn Gln Cys Cys His Asp |
| | | | | Glu Cys Ala Gly Gly Cys Ser Gly Pro Gln |
| | | | | Asp Thr Asp Cys Phe Ala Cys Arg His Phe |
| | | | | Asn Asp Ser Gly Ala Cys Val Pro Arg Cys |
| | | | | Pro Gln Pro Leu Val Tyr Asn Lys Leu Thr |
| | | | | Phe Gln Leu Glu Pro Asn Pro His Thr Lys |
| | | | | Tyr Gln Tyr Gly Gly Val Cys Val Ala Ser |
| | | | | Cys Pro His Asn Phe Val Val Asp Gln Thr |
| | | | | Ser Cys Val Arg Ala Cys Pro Pro Asp Lys |
| | | | | Met Glu Val Asp Lys Asn Gly Leu Lys Met |
| | | | | Cys Glu Pro Cys Gly Gly Leu Cys Pro Lys |
| | | | | Ala Cys Glu Gly Thr Gly Ser Gly Ser Arg |
| | | | | Phe Gln Thr Val Asp Ser Ser Asn Ile Asp |
| | | | | Gly Phe Val Asn Cys Thr Lys Ile Leu Gly |
| | | | | Asn Leu Asp Phe Leu Ile Thr Gln Gly Asp |
| | | | | Pro Trp His Lys Ile Pro Ala Leu Asp Pro |
| | | | | Glu Lys Leu Asn Val Phe Arg Thr Val Arg |
| | | | | Glu Ile Thr Gly Tyr Leu Asn Ile Gln Ser |
| | | | | Trp Pro Pro His Met His Asn Phe Ser Val |
| | | | | Phe Ser Asn Leu Thr Thr Ile Gly Gly Arg |
| | | | | Ser Leu Tyr Asn Arg Gly Phe Ser Leu Leu |
| | | | | Ile Met Lys Asn Leu Asn Val Thr Ser Leu |
| | | | | Gly Phe Arg Ser Leu Lys Glu Ile Ser Ala |
| | | | | Gly Arg Ile Tyr Ile Ser Ala Asn Arg Gln |
| | | | | Leu Cys Tyr His His Ser Leu Asn Trp Thr |
| | | | | Lys Val Leu Arg Gly Pro Thr Glu Glu Arg |
| | | | | Leu Asp Ile Lys His Asn Arg Pro Arg Arg |
| | | | | Asp Cys Val Ala Glu Gly Lys Val Cys Asp |
| | | | | Pro Leu Cys Ser Ser Gly Gly Cys Trp Gly |
| | | | | Pro Gly Pro Gly Gln Cys Leu Ser Cys Arg |
| | | | | Asn Tyr Ser Arg Gly Gly Val Cys Val Thr |
| | | | | His Cys Asn Phe Leu Asn Gly Glu Pro Arg |
| | | | | Glu Phe Ala His Glu Ala Glu Cys Phe Ser |
| | | | | Cys His Pro Glu Cys Gln Pro Met Glu Gly |
| | | | | Thr Ala Thr Cys Asn Gly Ser Gly Ser Asp |
| | | | | Thr Cys Ala Gln Cys Ala His Phe Arg Asp |
| | | | | Gly Pro His Cys Val Ser Ser Cys Pro His |
| | | | | Gly Val Leu Gly Ala Lys Gly Pro Ile Tyr |
| | | | | Lys Tyr Pro Asp Val Gln Asn Glu Cys Arg |
| | | | | Pro Cys His Glu Asn Cys Thr Gln Gly Cys |
| | | | | Lys Gly Pro Glu Leu Gln Asp Cys Leu Gly |
| | | | | Gln Thr Leu Val Leu Ile Gly Lys Thr His |
| | | | | Leu Thr Met Ala Leu Thr Val Ile Ala Gly |
| | | | | Leu Val Val Ile Phe Met Met Leu Gly Gly |
| | | | | Thr Phe Leu Tyr Trp Arg Gly Arg Arg Ile |
| | | | | Gln Asn Lys Arg Ala Met Arg Arg Tyr Leu |
| | | | | Glu Arg Gly Glu Ser Ile Glu Pro Leu Asp |
| | | | | Pro Ser Glu Lys Ala Asn Lys Val Leu Ala |
| | | | | Arg Ile Phe Lys Glu Thr Glu Leu Arg Ser |
| | | | | Leu Lys Val Leu Gly Ser Gly Val Phe Gly |
| | | | | Thr Val His Lys Gly Val Trp Ile Pro Glu |
| | | | | Gly Glu Ser Ile Lys Ile Pro Val Cys Ile |
| | | | | Lys Val Ile Glu Asp Lys Ser Gly Arg Gln |
| | | | | Ser Phe Gln Ala Val Thr Asp His Met Leu |
| | | | | Ala Ile Gly Ser Leu Asp His Ala His Ile |
| | | | | Val Arg Leu Leu Gly Leu Cys Pro Gly Ser |
| | | | | Ser Leu Gln Leu Val Thr Gln Tyr Leu Pro |
| | | | | Leu Gly Ser Leu Leu Asp His Val Arg Gln |
| | | | | His Arg Gly Ala Leu Gly Pro Gln Leu Leu |
| | | | | Leu Asn Trp Gly Val Gln Ile Ala Lys Gly |
| | | | | Met Tyr Tyr Leu Glu Glu His Gly Met Val |
| | | | | His Arg Asn Leu Ala Ala Arg Asn Val Leu |
| | | | | Leu Lys Ser Pro Ser Gln Val Gln Val Ala |
| | | | | Asp Phe Gly Val Ala Asp Leu Leu Pro Pro |
| | | | | Asp Asp Lys Gln Leu Leu Tyr Ser Glu Ala |
| | | | | Lys Thr Pro Ile Lys Trp Met Ala Leu Glu |
| | | | | Ser Ile His Phe Gly Lys Tyr Thr His Gln |
| | | | | Ser Asp Val Trp Ser Tyr Gly Val Thr Val |
| | | | | Trp Glu Leu Met Thr Phe Gly Ala Glu Pro |
| | | | | Tyr Ala Gly Leu Arg Leu Ala Glu Val Pro |
| | | | | Asp Leu Leu Glu Lys Gly Glu Arg Leu Ala |
| | | | | Gln Pro Gln Ile Cys Thr Ile Asp Val Tyr |
| | | | | Met Val Met Val Lys Cys Trp Met Ile Asp |
| | | | | Glu Asn Ile Arg Pro Thr Phe Lys Glu Leu |
| | | | | Ala Asn Glu Phe Thr Arg Met Ala Arg Asp |
| | | | | Pro Pro Arg Tyr Leu Val Ile Lys Arg Glu |
| | | | | Ser Gly Pro Gly Ile Ala Pro Gly Pro Glu |
| | | | | Pro His Gly Leu Thr Asn Lys Lys Leu Glu |
| | | | | Glu Val Glu Leu Glu Pro Glu Leu Asp Leu |
| | | | | Asp Leu Asp Leu Glu Ala Glu Glu Asp Asn |
| | | | | Leu Ala Thr Thr Thr Leu Gly Ser Ala Leu |
| | | | | Ser Leu Pro Val Gly Thr Leu Asn Arg Pro |
| | | | | Arg Gly Ser Gln Ser Leu Leu Ser Pro Ser |
| | | | | Ser Gly Tyr Met Pro Met Asn Gln Gly Asn |
| | | | | Leu Gly Glu Ser Cys Gln Glu Ser Ala Val |
| | | | | Ser Gly Ser Ser Glu Arg Cys Pro Arg Pro |
| | | | | Val Ser Leu His Pro Met Pro Arg Gly Cys |
| | | | | Leu Ala Ser Glu Ser Ser Glu Gly His Val |
| | | | | Thr Gly Ser Glu Ala Glu Leu Gln Glu Lys |
| | | | | Val Ser Met Cys Arg Ser Arg Ser Arg Ser |
| | | | | Arg Ser Pro Arg Pro Arg Gly Asp Ser Ala |
| | | | | Tyr His Ser Gln Arg His Ser Leu Leu Thr |
| | | | | Pro Val Thr Pro Leu Ser Pro Pro Gly Leu |
| | | | | Glu Glu Glu Asp Val Asn Gly Tyr Val Met |
| | | | | Pro Asp Thr His Leu Lys Gly Thr Pro Ser |
| | | | | Ser Arg Glu Gly Thr Leu Ser Ser Val Gly |
| | | | | Leu Ser Ser Val Leu Gly Thr Glu Glu Glu |
| | | | | Asp Glu Asp Glu Glu Tyr Glu Tyr Met Asn |
| | | | | Arg Arg Arg Arg His Ser Pro Pro His Pro |
| | | | | Pro Arg Pro Ser Ser Leu Glu Glu Leu Gly |
| | | | | Tyr Glu Tyr Met Asp Val Gly Ser Asp Leu |
| | | | | Ser Ala Ser Leu Gly Ser Thr Gln Ser Cys |
| | | | | Pro Leu His Pro Val Pro Ile Met Pro Thr |
| | | | | Ala Gly Thr Thr Pro Asp Glu Asp Tyr Glu |
| | | | | Tyr Met Asn Arg Gln Arg Asp Gly Gly Gly |
| | | | | Pro Gly Gly Asp Tyr Ala Ala Met Gly Ala |
| | | | | Cys Pro Ala Ser Glu Gln Gly Tyr Glu Glu |
| | | | | Met Arg Ala Phe Gln Gly Pro Gly His Gln |
| | | | | Ala Pro His Val His Tyr Ala Arg Leu Lys |
| | | | | Thr Leu Arg Ser Leu Glu Ala Thr Asp Ser |
| | | | | Ala Phe Asp Asn Pro Asp Tyr Trp His Ser |
| | | | | Arg Leu Phe Pro Lys Ala Asn Ala Gln Arg |
| | | | | Thr |
| 12 | Heavy Chain of | human | PROTEIN | 1 EVQLLESGGG LVQPGGSLRL SCAASGFTFS |
| | Antibody A | heavy | | HYVMAWVRQA PGKGLEWVSS |
| | | chain | | 51 ISSSGGWTLY ADSVKGRFTI SRDNSKNTLY |
| | | | | LQMNSLRAED TAVYYCTRGL |
| | | | | 101 KMATIFDYWG QGTLVTVSSA STKGPSVFPL |
| | | | | APCSRSTSES TAALGCLVKD |
| | | | | 151 YFPEPVTVSW NSGALTSGVH TFPAVLQSSG |
| | | | | LYSLSSVVTV PSSNFGTQTY |
| | | | | 201 TCNVDHKPSN TKVDKTVERK CCVECPPCPA |
| | | | | PPVAGPSVFL FPPKPKDTLM |
| | | | | 251 ISRTPEVTCV VVDVSHEDPE VQFNWYVDGV |
| | | | | EVHNAKTKPR EEQFNSTFRV |
| | | | | 301 VSVLTVVHQD WLNGKEYKCK VSNKGLPAPI |
| | | | | EKTISKTKGQ PREPQVYTLP |
| | | | | 351 PSREEMTKNQ VSLTCLVKGF YPSDIAVEWE |
| | | | | SNGQPENNYK TTPPMLDSDG |
| | | | | 401 SFFLYSKLTV DKSRWQQGNV FSCSVMHEAL |
| | | | | HNHYTQKSLS LSPGK |
| 13 | Light Chain of | light | PROTEIN | 1 QSALTQPASV SGSPGQSITI SCTGTSSDVG |
| | Antibody A | heavy | | SYNVVSWYQQ HPGKAPKLII |
| | | chain | | 51 YEVSQRPSGV SNRFSGSKSG NTASLTISGL |
| | | | | QTEDEADYYC CSYAGSSIFV |
| | | | | 101 IFGGGTKVTV LGQPKAAPSV TLFPPSSEEL |
| | | | | QANKATLVCL VSDFYPGAVT |
| | | | | 151 VAWKADGSPV KVGVETTKPS KQSNNKYAAS |
| | | | | SYLSLTPEQW KSHRSYSCRV |
| | | | | 201 THEGSTVEKT VAPAECS |

1. A method of treating advanced gynecological cancer in a human patient comprising administering to the patient, an effective amount of (a) an anti-ErbB3 antibody comprising CDRH1, CDRH2, and CDRH3 sequences comprising the amino acid sequences set forth in SEQ ID NO: 5 (CDRH1), SEQ ID NO: 6 (CDRH2), and SEQ ID NO: 7 (CDRH3), and CDRL1, CDRL2, and CDRL3 sequences comprising the amino acid sequences set forth in SEQ ID NO: 8 (CDRL1), SEQ ID NO: 9 (CDRL2), and SEQ ID NO: 10 (CDRL3), and (b) paclitaxel, wherein the method comprises at least one cycle, wherein the cycle is a period of 4 weeks, wherein for each cycle the anti-ErbB3 antibody is administered at a weekly dose of 20 mg/kg and the paclitaxel is administered at a dose of 80 mg/m² once per week, and wherein the gynecological cancer is selected from the group consisting of locally advanced or metastatic epithelial ovarian cancer, recurrent ovarian cancer, fallopian tube cancer and primary peritoneal cancer.

2-4. (canceled)

5. A method of treating advanced gynecological cancer in a human patient comprising administering to the patient, an effective amount of (a) an anti-ErbB3 antibody comprising CDRH1, CDRH2, and CDRH3 sequences comprising the amino acid sequences set forth in SEQ ID NO: 5 (CDRH1), SEQ ID NO: 6 (CDRH2), and SEQ ID NO: 7 (CDRH3), and CDRL1, CDRL2, and CDRL3 sequences comprising the amino acid sequences set forth in SEQ ID NO: 8 (CDRL1), SEQ ID NO: 9 (CDRL2), and SEQ ID NO: 10 (CDRL3), and (b) paclitaxel, wherein the method comprises at least one cycle, wherein the cycle is a period of 4 weeks, wherein for each cycle the anti-ErbB3 antibody is administered every other week at a dose of 40 mg/kg and the paclitaxel is administered at a dose of 80 mg/m² once per week, and wherein the gynecological cancer is selected from the group consisting of locally advanced or metastatic epithelial ovarian cancer, recurrent ovarian cancer, fallopian tube cancer and primary peritoneal cancer.

6. The method of claim 1, wherein the patient has been treated previously with a platinum-based compound.

7. The method of claim 1, wherein the cancer is platinum-resistant or refractory.

8. The method of claim 1, wherein the cancer is resistant/refractory to cisplatin.

9. The method of claim 1, wherein the anti-ErbB3 antibody comprises heavy and light chain variable regions comprising the amino acid sequences set forth in SEQ ID NOs:2 and 4, respectively.

10. The method of claim 1, wherein paclitaxel is administered immediately following the anti-ErbB3 antibody.

11. The method of claim 1, wherein the patient is pretreated with an agent that prevents hypersensitivity prior to paclitaxel administration.

12. The method of claim 11, wherein the agent that prevents hypersensitivity is selected from the group consisting of: 20 mg of dexamethasone; 50 mg of diphenhydramine; 300 mg or cimetidine; and 50 mg of ranitidine.

13. The method of claim 1, wherein the patient does not have metastatic disease.

14. The method of claim 1, wherein the patient has metastatic disease.

15. The method of claim 1, wherein the treatment produces at least one therapeutic effect selected from the group consisting of reduction in size of a tumor, reduction in number of metastatic lesions over time, complete response, partial response, stable disease, increase in overall response rate, or a pathologic complete response.

16. A combination for use in treating platinum resistant or refractory gynecological cancer in a human patient, the combination comprising a clinically proven safe and effective amount of (a) an anti-ErbB3 antibody comprising CDRH1, CDRH2, and CDRH3 sequences comprising the amino acid sequences set forth in SEQ ID NO: 5 (CDRH1) SEQ ID NO: 6 (CDRH2) and SEQ ID NO: 7 (CDRH3), and CDRL1, CDRL2, and CDRL3 sequences comprising the amino acid sequences set forth in SEQ ID NO: 8 (CDRL1) SEQ ID NO: 9 (CDRL2) and SEQ ID NO: 10 (CDRL3) and (b) paclitaxel.

17. The combination of claim 16, wherein the anti-ErbB3 antibody comprises heavy and light chain variable regions comprising the amino acid sequences set forth in SEQ ID NOs:2 and 4, respectively.

18. The combination of claim 16, wherein the antibody is formulated for intravenous administration at a dose of 20 mg/kg.

19. A kit comprising a dose of an anti-ErbB3 antibody comprising CDRH1, CDRH2, and CDRH3 sequences comprising the amino acid sequences set forth, respectively, in SEQ ID NO: 5 (CDRH1) SEQ ID NO: 6 (CDRH2) and SEQ ID NO: 7 (CDRH3), and CDRL1, CDRL2, and CDRL3 sequences comprising the amino acid sequences set forth, respectively, in SEQ ID NO: 8 (CDRL1) SEQ ID NO: 9 (CDRL2) and SEQ ID NO: 10 (CDRL3), and instructions for using the anti-ErbB3 antibody in the method of claim 1.

20. The kit of claim 19, said kit comprising at least 500 mg of the antibody.

21. The kit of claim 19, said kit comprising at least 1 mg of paclitaxel.

22. An anti-ErbB3 antibody comprising SEQ ID NO: 5 (CDRH1), SEQ ID NO: 6 (CDRH2), SEQ ID NO: 7 (CDRH3), SEQ ID NO: 8 (CDRL1), SEQ ID NO: 9 (CDRL2), and SEQ ID NO: 10 (CDRL3), for co-administration with paclitaxel in at least one cycle, wherein the cycle is a period of 4 weeks, and wherein for each cycle the anti-ErbB3 antibody is administered every other week at a dose of 40 mg/kg and the paclitaxel is administered at a weekly dose of 80 mg/m².

23. The method of claim 1, wherein for each cycle the anti-ErbB3 antibody is administered at a weekly dose of 20 mg/kg, except for cycle 1 week 1 wherein the anti-ErbB3 antibody optionally may be administered at 40 mg/kg.
